(19) United States
(12) Patent Application Publication
(10) Pub. No.: US 2017/0354722 A1
(43) Pub. Date: Dec. 14, 2017

(54) COMPOSITIONS AND METHODS FOR TREATING POST-OPERATIVE COMPLICATIONS OF CARDIOPULMONARY SURGERY

(71) Applicant: MOR RESEARCH APPLICATIONS LTD.

(72) Inventor: Dan ABRAMOV, Lehavim (IL)

(21) Appl. No.: 15/524,380

(22) PCT Filed: Nov. 9, 2015
(86) PCT No.: PCT/IB15/02213

§ 371 Date: May 4, 2017

Related U.S. Application Data
(60) Provisional application No. 62/076,923, filed on Nov. 7, 2014.

(51) Int. Cl. A61K 38/57

(52) U.S. Cl. CPC A61K 38/57

(57) ABSTRACT

Compositions and methods for treating damage inflicted by use of a cardio-pulmonary bypass (CPB) machine, particularly excessive bleeding and multi organ failure, by administering a pharmaceutical composition comprising alpha-1 antitrypsin (AAT-1).

### RELATED APPLICATIONS

This application claims the priority of U.S. provisional application Ser. No. U.S. Ser. No. 62/076,923, entitled “COMPOSITIONS AND METHODS FOR TREATING POST-OPERATIVE COMPLICATIONS OF CARDIOPULMONARY SURGERY,” filed Nov. 7, 2014, which is incorporated herein by reference in its entirety for all purposes.

### FIELD OF INVENTION

The instant invention is related to compositions and methods for treating post-operative complications of cardiopulmonary surgery.

### BACKGROUND

Open heart surgery using cardiopulmonary bypass (CPB) is one of the most common surgical procedures performed today. Approximately 1,000,000 operations are conducted worldwide each year, of which 500,000 are conducted in the United States alone. Use of CPB can profoundly alter haemostasis as well as injure vital organs, predisposing patients to major haemorrhagic complications and multi organ failure.

Excessive post-operative bleeding necessitating additional surgery occurs in 7% of patients undergoing CPB. Re-operation for bleeding increases hospital mortality, substantially increases post-operative hospital stay and has a sizeable effect on health care costs.

### BRIEF SUMMARY OF INVENTION

In some embodiments, the present invention is a method of treatment, comprising: administering at least one dose of alpha-1 antitrypsin (AAT-1) at a concentration ranging from 60 mg/kg body weight to 120 mg/kg body weight to a patient having a coronary surgery with a cardiopulmonary bypass (CPB) so as to result in a reduced CPB-inflicted organ injury.

In some embodiments, the present invention is a method of treatment, comprising: administering at least one dose of alpha-1 antitrypsin (AAT-1) at a concentration ranging from 60 mg/kg body weight to 100 mg/kg body weight to a patient having a coronary surgery with a cardiopulmonary bypass (CPB) so as to result in a reduced CPB-inflicted organ injury.

In some embodiments, the at least one dose of AAT-1 is administered using intravenous, intranasal, or via the cardiopulmonary bypass machine reservoir. In some embodiments, the at least one dose of AAT-1 is administered using intravenous administration, intranasal administration, or administration via the cardiopulmonary bypass machine reservoir. In some embodiments, the at least one dose of AAT-1 is administered using intravenous administration. In some embodiments, the at least one dose of AAT-1 is administered using intranasal administration. In some embodiments, the at least one dose of AAT-1 is administered using a cardiopulmonary bypass machine reservoir.

In some embodiments, the present invention is a method of treatment, comprising: administering at least one dose of AAT-1 at a concentration ranging from 60 mg/kg body weight to 120 mg/kg body weight to a patient having a coronary surgery so as to result in a reduction of post-operative bleeding in the patient.

In some embodiments, the present invention is a method of treatment, comprising: administering at least one dose of AAT-1 at a concentration ranging from 60 mg/kg body weight to 100 mg/kg body weight to a patient having a coronary surgery so as to result in a reduction of post-operative bleeding in the patient.

In some embodiments, the present invention is a method of treatment, comprising: administering a preoperative therapeutically effective amount of AAT-1 to a patient having a coronary surgery with a CPB so as to result in a reduced CPB-inflicted organ injury.

### BRIEF DESCRIPTION OF THE DESCRIBED SEQUENCES

The nucleic and/or amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listings are as follows:

SEQ ID NO. 1 is a cDNA sequence of human alpha-1 antitrypsin transcript that can be used in some embodiments of the inventive method of the present invention:

GAATTCCAGGTTGGAGGGGCGGCAACCTCCTGCCAGCCTTCAGGCCACTC
TCCTGTGCCTGCCAGAAGAGACAGAGCTTGAGGAGAGCTTGAGGAGAGCA
GGAAAGGTGGAACATTGCTGCTGCTGCTCACTCAGTTCCACAGGTGGGAG
GAACAGCAGGGCTTAGAGTGGGGGTCATTGTGCAGATGGGAAAACAAAGG
CCCAGAGAGGGGAAGAAATGCCTAGGAGCTACCGAGGGCAGGCGACCTCA
ACCACAGCCCAGTGCTGGAGCTGTGAGTGGATGTAGAGCAGCGGAATATC
CATTCAGCCAGCTCAGGGGAAGGACAGGGGCCCTGAAGCCAGGGGATGGA
GCTGCAGGGAAGGGAGCTCAGAGAGAAGGGGAGGGGAGTCTGAGCTCAGT
TTCCCGCTGCCTGAAAGGAGGGTGGTACCTACTCCCTTCACAGGGTAACT
GAATGAGAGACTGCCTGGAGGAAAGCTCTTCAAGTGTGGCCCACCCCACC
CCAGTGACACCAGCCCCTGACACGGGGGAGGGAGGGCAGCATCAGGAGGG
GCTTTCTGGGCACACCCAGTACCCGTCTCTGAGCTTTCCTTGAACTGTTG
CATTTTAATCCTCACAGCAGCTCAACAAGGTACATACCGTCACCATCCCC
ATTTTACAGATAGGGAAATTGAGGCTCGGAGCGGTTAAACAACTCACCTG
AGGCCTCACAGCCAGTAAGTGGGTTCCCTGGTCTGAATGTGTGTGCTGGA
GGATCCTGTGGGTCACTCGCCTGGTAGAGCCCCAAGGTGGAGGCATAAAT
GGGACTGGTGAATGACAGAAGGGGCAAAAATGCACTCATCCATTCACTCT
GCAAGTATCTACGGCACGTACGCCAGCTCCCAAGCAGGTTTGCGGGTTGC
ACAGCGGAGCGATGCAATCTGATTTAGGCTTTTAAAGGATTGCAATCAAG
TGGGACCCACTAGCCTCAACCCTGTACCTCCCCTCCCCTCCACCCCCAG
C...

SEQ ID NO. 2 is an amino acid sequence of human alpha −1 antitrypsin protein that can be used in some embodiments of the inventive method of the present invention:

MPSSVSWGIL LLAGLCCLVP VSLAEDPQGD AAQKTDTSHH
DQDHPTFNKI TPNLAEFAFS LYRQLAHQSN STNIFFSPVS
IATAFAMLSL GTKADTHDEI LEGLNFNLTE IPEAQIHEGF
QELLRTLNQP DSQLQLTTGN GLFLSEGLKL VDKFLEDVKK
LYHSEAFTVN FGDTEEAKKQ INDYVEKGTQ GKIVDLVKEL
DRDTVFALVN YIFFKGKWER PFEVKDTEEE DFHVDQVTTV
KVPMMKRLGM FNIQHCKKLS SWVLLMKYLG NATAIFFLPD
EGKLQHLENE LTHDIITKFL ENEDRRSASL HLPKLSITGT
YDLKSVLGQL GITKVFSNGA DLSGVTEEAP LKLSKAVHKA
VLTIDEKGTE AAGAMFLEAI PMSIPPEVKF NKPFVFLMIE
QNTKSPLFMG KVVNPTQK

### DETAILED DESCRIPTION

### I. Abbreviations

AAT-1 Alpha-1 Anti Trypsin
ACT Activated Clotting Time
AM Acute Kidney Injury
BAL Broncho alveolar lavage
CABG Coronary Artery Bypass Grafting
CPB Cardio Pulmonary Bypass
CVP Central Venous Pressure
HBV Hepatitis B Virus
HCV Hepatitis C Virus
MM Kidney injury molecule
MI Myocardial Infarction
N-GAL Neutrophil gelatinase

### II. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

As used herein, “abnormal” refers to a deviation from normal characteristics. Normal characteristics can be found in a control, a standard for a population, etc. For example, where the abnormal condition is an injury or physical response, such as injury resultant from cardiac surgery employing cardiopulmonary bypass, a few appropriate sources of normal characteristics might include an individual or a population standard of a collection of individuals who are not suffering from the injury or experiencing the particular physical response. Controls or standards appropriate for comparison to a sample, for the determination of abnormality, include samples believed to be normal as well as laboratory determined values, even though such values are possibly arbitrarily set, and keeping in mind that such values may vary from laboratory to laboratory. Laboratory standards and values may be set based on a known or determined population value and may be supplied in the format of a graph or table that permits easy comparison of measured, experimentally determined values.

As used herein, “administration” refers to the introduction of a composition into a subject by a chosen route. Administration of an active compound or composition can be by any route known to one of skill in the art. Administration can be local or systemic. Local administration includes routes of administration typically used for systemic administration, for example by directing intravascular administration to the arterial supply for a particular organ. Thus, in some embodiments, local administration includes intra-arterial administration and intravenous administration when such administration is targeted to the vasculature supplying a particular organ. Local administration also includes the incorporation of active compounds and agents into implantable devices or constructs, such as vascular stents or other reservoirs, which release the active agents and compounds over extended time intervals for sustained treatment effects.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

As used herein, “cardiac surgery” refers to any surgical procedure involving treatment of the cardiovascular or respiratory system of a subject, and which can impair or temporarily stop normal cardiovascular function. In particular examples, cardiac surgery requires the heart of the subject to be stopped, but this is not an absolute requirement of all forms of cardiac surgery. Particular examples of cardiac surgery include coronary artery bypass grafting surgery, aortic valve replacement or repair, mitral valve replacement or repair, tricuspid valve replacement or repair, ascending aorta replacement, heart transplantation, lung transplantation, usage of extracorporeal membrane oxygenation (ECMO) machine or any combination of the above.

As used herein, “cardiopulmonary bypass” or “CPB” refers to a surgical technique for maintaining blood circulation and oxygenation when heart function is impaired or temporarily stopped. CPB is achieved through use of a pump. In particular examples, the pump is known as a “heart-lung machine,” CPB pump, or CPB machine. In other examples, a type CPB is also known as “extracorporeal membrane perfusion.”

As used herein, “functional fragments” and “variants of a polypeptide” include those fragments and variants that maintain one or more functions of the parent polypeptide, such as a functional fragment or variant of AAT-1. It is recognized that the gene or cDNA encoding a polypeptide can be considerably mutated without materially altering one or more the polypeptide's functions. First, the genetic code is well-known to be degenerate, and thus different codons encode the same amino acids. Second, even where an amino acid substitution is introduced, the mutation can be conservative and have no material impact on the essential functions of a protein. Third, part of a polypeptide chain can be deleted without impairing or eliminating all of its functions. Fourth, insertions or additions can be made in the polypeptide chain for example, adding epitope tags, without impairing or eliminating its functions. Other modifications that can be made without materially impairing one or more functions of a polypeptide include, for example, in vivo or in vitro chemical and biochemical modifications or the incorporation of unusual amino acids. Such modifications include, for example, acetylation, carboxylation, phosphorylation, glycosylation, ubiquination, labeling, e.g., with radionucleides, and various enzymatic modifications.

Conservative amino acid substitution tables providing functionally similar amino acids are well known to one of ordinary skill in the art. The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Variant amino acid sequences may, for example, be 80%, 85%, 90% or even 95% or 98% identical to the native AAT-1 amino acid sequence. Programs and algorithms for determining percentage identity can be found at the NCBI website.

As used herein, an “injectable composition” refers to a pharmaceutically acceptable fluid composition comprising at least one active ingredient, for example, a protein, peptide, or antibody. The active ingredient is usually dissolved or suspended in a physiologically acceptable carrier, and the composition can additionally comprise minor amounts of one or more non-toxic auxiliary substances, such as emulsifying agents, preservatives, pH buffering agents and the like. Such injectable compositions that are useful for use with the compositions of this disclosure are conventional; appropriate formulations are well known in the art.

As used herein, an “organ injury” refers to an impairment of normal organ function in a mammalian subject, including human and veterinary subjects. Organ injury as understood herein does not require complete loss of organ function. In particular examples, loss of specific organ function is diagnosed by detection of biological markers and/or other experimental methods. Examples include, but are not limited to, detection of liver enzymes (e.g., abnormal levels) to indicate liver injury, increased bleeding/blood loss, BBB disruption, deterioration in neuropsychological tests, detection of postoperative inflammatory markers (e.g., increased levels of IL-6, TNF-alpha, IL-1 beta, IL-8, MCP-1, LDH, and D dimmer), detection of abnormal pulmonary function (e.g., by measuring AaDO₂ and lung mechanics) decrease in renal function (e.g., by measuring AKI markers) or any combination thereof. As used herein, “CPB-inflicted organ injury” refers to organ injury resulting in a patient postoperatively, after a CPB was used on the patient.

As used herein, “pharmaceutically acceptable carrier(s)” refer to the pharmaceutically acceptable carrier(s) useful in this disclosure are conventional. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, a “pharmaceutical agent” refers to a chemical compound or composition capable of inducing a desired therapeutic or prophylactic effect when properly administered to a subject.

As used herein, “preventing an injury” or “treating an injury” refers to inhibiting the full development of an injury or pathological condition, for example inhibiting excessive post-operative bleeding or organ injury in a person who has or is undergoing cardiac surgery. Treatment refers to a therapeutic intervention that ameliorates a sign or symptom of the injury or pathological condition after it has begun to develop.

As used herein, “resultant” or “result” refers to an effect of a causative event is said to be “resultant” from that event. For example, in particular subjects, excessive bleeding is resultant from use of cardiopulmonary bypass in cardiac surgery. In particular examples, a resultant effect may immediately follow the causative event. In other examples, a resultant effect develops as a delayed response to the event. For example, certain organ damage may be resultant from use of cardiopulmonary bypass on a subject during cardiac surgery, but the extent of the damage may not be fully apparent for hours or even days after the surgery.

As used herein, a “subject” refers to a living multi-cellular organism, including vertebrate organisms, a category that includes both human and non-human mammals.

As used herein, a “therapeutically effective amount” refers to a quantity of compound sufficient to achieve a desired effect in a subject being treated. An effective amount of a compound may be administered in a single dose, or in several doses, for example daily, during a course of treatment. However, the effective amount will be dependent on the compound applied, the subject being treated, the severity and type of the affliction, and the manner of administration of the compound.

### III. Overview of Several Embodiments

Described herein are compositions including a therapeutically effective amount of alpha-1 antitrypsin (AAT-1), or a functional variant thereof, for use in preventing or treating injury to a subject during or resultant from cardiac surgery, and particularly from the use of cardiopulmonary bypass. In some embodiments, the injury is excessive post-operative bleeding or organ injury.

In some embodiments, the present invention is a method of treatment, comprising: administering at least one dose of alpha-1 antitrypsin (AAT-1) at a concentration ranging from 60 mg/kg body weight to 100 mg/kg body weight to a patient having a coronary surgery with a cardiopulmonary bypass (CPB) so as to result in a reduced CPB-inflicted organ injury.

In some embodiments, the present invention is a method of treatment, comprising: administering at least one dose of alpha-1 antitrypsin (AAT-1) at a concentration ranging from 60 mg/kg body weight to 120 mg/kg body weight to a patient having a coronary surgery with a cardiopulmonary bypass (CPB) so as to result in a reduced CPB-inflicted organ injury.

In some embodiments, the present invention is a method of treatment, comprising: administering at least one dose of AAT-1 at a concentration ranging from 60 mg/kg body weight to 100 mg/kg body weight to a patient having a coronary surgery so as to result in a reduction of post-operative bleeding in the patient.

In some embodiments, the present invention is a method of treatment, comprising: administering at least one dose of AAT-1 at a concentration ranging from 60 mg/kg body weight to 120 mg/kg body weight to a patient having a coronary surgery so as to result in a reduction of post-operative bleeding in the patient.

In some embodiments, the present invention is a method of treatment, comprising: administering a preoperative therapeutically effective amount of AAT-1 to a patient having a coronary surgery with a CPB so as to result in a reduced CPB-inflicted organ injury.

In some embodiments, the present invention is a method of treatment, comprising: administering at least one dose of alpha-1 antitrypsin (AAT-1) at a concentration ranging from 60 mg/kg body weight to 100 mg/kg body weight to a patient having a cardiac surgery using a cardiopulmonary bypass (CPB) so as to result in a reduced CPB-inflicted organ injury.

In some embodiments, the present invention is a method of treatment, comprising: administering at least one dose of alpha-1 antitrypsin (AAT-1) at a concentration ranging from 60 mg/kg body weight to 120 mg/kg body weight to a patient having a cardiac surgery using a cardiopulmonary bypass (CPB) so as to result in a reduced CPB-inflicted organ injury.

In some embodiments, the present invention is a method of treatment, comprising: administering at least one dose of AAT-1 at a concentration ranging from 60 mg/kg body weight to 100 mg/kg body weight to a patient having a cardiac surgery using a cardiopulmonary bypass (CPB) so as to result in a reduction of post-operative bleeding in the patient.

In some embodiments, the present invention is a method of treatment, comprising: administering at least one dose of AAT-1 at a concentration ranging from 60 mg/kg body weight to 120 mg/kg body weight to a patient having a cardiac surgery using a cardiopulmonary bypass (CPB) so as to result in a reduction of post-operative bleeding in the patient.

In some embodiments, the present invention is a method of treatment, comprising: administering a preoperative therapeutically effective amount of AAT-1 to a patient having a cardiac surgery using a CPB so as to result in a reduced CPB-inflicted organ injury. In some embodiments, the at least one dose of AAT-1 is administered to the patient between 2-5 hours before the coronary surgery.

In some embodiments, the present invention is a method of treatment, where AAT-1 administration reduces postoperative BBB disruption by 50%. In some embodiments, AAT-1 administration reduces postoperative BBB disruption by 20%-80%. In some embodiments, AAT-1 administration reduces postoperative BBB disruption by 30%-80%. In some embodiments, AAT-1 administration reduces postoperative BBB disruption by 40%-80%. In some embodiments, AAT-1 administration reduces postoperative BBB disruption by 50%-80%. In some embodiments, AAT-1 administration reduces postoperative BBB disruption by 60%-80%. In some embodiments, AAT-1 administration reduces postoperative BBB disruption by 70%-80%. In some embodiments, AAT-1 administration reduces postoperative BBB disruption by 20%-70%. In some embodiments, AAT-1 administration reduces postoperative BBB disruption by 20%-60%. In some embodiments, AAT-1 administration reduces postoperative BBB disruption by 20%-50%. In some embodiments, AAT-1 administration reduces postoperative BBB disruption by 20%-40%. In some embodiments, AAT-1 administration reduces postoperative BBB disruption by 20%-30%.

In some embodiments, the present invention is a method of treatment, where AAT-1 administration improves postoperative cognitive function by 50% compared to a patient not having been administered AAT-1. In some embodiments, AAT-1 administration improves postoperative cognitive function by 30-70% compared to a patient not having been administered AAT-1. In some embodiments, AAT-1 administration improves postoperative cognitive function by 4-70% compared to a patient not having been administered AAT-1. In some embodiments, AAT-1 administration improves postoperative cognitive function by 50-70% compared to a patient not having been administered AAT-1. In some embodiments, AAT-1 administration improves postoperative cognitive function by 60-70% compared to a patient not having been administered AAT-1. In some embodiments, AAT-1 administration improves postoperative cognitive function by 30-60% compared to a patient not having been administered AAT-1. In some embodiments, AAT-1 administration improves postoperative cognitive function by 30-50% compared to a patient not having been administered AAT-1. In some embodiments, AAT-1 administration improves postoperative cognitive function by 30-40% compared to a patient not having been administered AAT-1.

In some embodiments, the present invention is a method of treatment, where AAT-1 administration reduces postoperative cognitive decline by 50%. In some embodiments, the present invention is a method of treatment, where AAT-1 administration reduces postoperative cognitive decline by 30-70%. In some embodiments, the present invention is a method of treatment, where AAT-1 administration reduces postoperative cognitive decline by 40-70%. In some embodiments, the present invention is a method of treatment, where AAT-1 administration reduces postoperative cognitive decline by 50-70%. In some embodiments, the present invention is a method of treatment, where AAT-1 administration reduces postoperative cognitive decline by 60-70%. In some embodiments, the present invention is a method of treatment, where AAT-1 administration reduces postoperative cognitive decline by 30-60%. In some embodiments, the present invention is a method of treatment, where AAT-1 administration reduces postoperative cognitive decline by 30-50%. In some embodiments, the present invention is a method of treatment, where AAT-1 administration reduces postoperative cognitive decline by 30-40%.

In some embodiments, the at least one dose of AAT-1 is administered to the patient between 2-12 hours before the coronary surgery. In some embodiments, the at least one dose of AAT-1 is administered to the patient between 2-11 hours before the coronary surgery. In some embodiments, the at least one dose of AAT-1 is administered to the patient between 2-10 hours before the coronary surgery. In some embodiments, the at least one dose of AAT-1 is administered to the patient between 2-9 hours before the coronary surgery. In some embodiments, the at least one dose of AAT-1 is administered to the patient between 2-8 hours before the coronary surgery. In some embodiments, the at least one dose of AAT-1 is administered to the patient between 2-7 hours before the coronary surgery. In some embodiments, the at least one dose of AAT-1 is administered to the patient between 2-6 hours before the coronary surgery. In some embodiments, the at least one dose of AAT-1 is administered to the patient between 2-5 hours before the coronary surgery. In some embodiments, the at least one dose of AAT-1 is administered to the patient between 2-4 hours before the coronary surgery. In some embodiments, the at least one dose of AAT-1 is administered to the patient between 2-3 hours before the coronary surgery.

In some embodiments, the at least one dose of AAT-1 is administered to the patient between 3-12 hours before the coronary surgery. In some embodiments, the at least one dose of AAT-1 is administered to the patient between 4-12 hours before the coronary surgery. In some embodiments, the at least one dose of AAT-1 is administered to the patient between 5-12 hours before the coronary surgery. In some embodiments, the at least one dose of AAT-1 is administered to the patient between 6-12 hours before the coronary surgery. In some embodiments, the at least one dose of AAT-1 is administered to the patient between 7-12 hours before the coronary surgery. In some embodiments, the at least one dose of AAT-1 is administered to the patient between 8-12 hours before the coronary surgery. In some embodiments, the at least one dose of AAT-1 is administered to the patient between 9-12 hours before the coronary surgery. In some embodiments, the at least one dose of AAT-1 is administered to the patient between 10-12 hours before the coronary surgery. In some embodiments, the at least one dose of AAT-1 is administered to the patient between 11-12 hours before the coronary surgery.

In some embodiments, the at least one dose of AAT-1 is administered to the patient between 2-12 hours before the coronary surgery. In some embodiments, the at least one dose of AAT-1 is administered to the patient between 3-11 hours before the coronary surgery. In some embodiments, the at least one dose of AAT-1 is administered to the patient between 4-10 hours before the coronary surgery. In some embodiments, the at least one dose of AAT-1 is administered to the patient between 5-9 hours before the coronary surgery. In some embodiments, the at least one dose of AAT-1 is administered to the patient between 6-8 hours before the coronary surgery.

In some embodiments, if AAT-1 is administered to the patient in a time frame less than 2 hours, the patient may become hypersensitive. In some embodiments, AAT-1 can be administered up to twelve hours before the coronary surgery because AAT-1 is degraded slowly in the plasma.

In some embodiments, the composition can be administered to the subject before the cardiac surgery, during the cardiac surgery, after the cardiac surgery, and/or a combination thereof. In some embodiments, the composition is administered to the subject in multiple doses. In some embodiments, the composition is administered to the subject in a single dose.

In some embodiments of the described composition, the concentration of AAT-1, or the functional variant thereof, is 1 gram in 50 cc sterile fluid in the form of a sterile or physiologically isotonic aqueous solution.

In some embodiments of the methods of the present invention, at least one dose of AAT-1 is administered to a patient having a coronary surgery with a cardiopulmonary bypass at a concentration ranging from 30-300 mg/kg body weight. In some embodiments, at least one dose of AAT-1 is administered to a patient having a coronary surgery with a cardiopulmonary bypass at a concentration ranging from 30-250 mg/kg body weight. In some embodiments, at least one dose of AAT-1 is administered to a patient having a coronary surgery with a cardiopulmonary bypass at a concentration ranging from 30-200 mg/kg body weight. In some embodiments, at least one dose of AAT-1 is administered to a patient having a coronary surgery with a cardiopulmonary bypass at a concentration ranging from 30-150 mg/kg body weight. In some embodiments, at least one dose of AAT-1 is administered to a patient having a coronary surgery with a cardiopulmonary bypass at a concentration ranging from 30-125 mg/kg body weight.

In some embodiments, at least one dose of AAT-1 is administered to a patient having a coronary surgery with a cardiopulmonary bypass at a concentration ranging from 50-300 mg/kg body weight. In some embodiments, at least one dose of AAT-1 is administered to a patient having a coronary surgery with a cardiopulmonary bypass at a concentration ranging from 100-300 mg/kg body weight. In some embodiments, at least one dose of AAT-1 is administered to a patient having a coronary surgery with a cardiopulmonary bypass at a concentration ranging from 125-300 mg/kg body weight. In some embodiments, at least one dose of AAT-1 is administered to a patient having a coronary surgery with a cardiopulmonary bypass at a concentration ranging from 150-300 mg/kg body weight. In some embodiments, at least one dose of AAT-1 is administered to a patient having a coronary surgery with a cardiopulmonary bypass at a concentration ranging from 200-300 mg/kg body weight. In some embodiments, at least one dose of AAT-1 is administered to a patient having a coronary surgery with a cardiopulmonary bypass at a concentration ranging from 250-300 mg/kg body weight.

In some embodiments, at least one dose of AAT-1 is administered to a patient having a coronary surgery with a cardiopulmonary bypass at a concentration ranging from 50-250 mg/kg body weight. In some embodiments, at least one dose of AAT-1 is administered to a patient having a coronary surgery with a cardiopulmonary bypass at a concentration ranging from 100-200 mg/kg body weight. In some embodiments, at least one dose of AAT-1 is administered to a patient having a coronary surgery with a cardiopulmonary bypass at a concentration ranging from 125-175 mg/kg body weight.

In some embodiments, at least one dose of AAT-1 is administered to a patient having a coronary surgery with a cardiopulmonary bypass at a concentration ranging from 30-100 mg/kg body weight. In some embodiments, at least one dose of AAT-1 is administered to a patient having a coronary surgery with a cardiopulmonary bypass at a concentration ranging from 30-90 mg/kg body weight. In some embodiments, at least one dose of AAT-1 is administered to a patient having a coronary surgery with a cardiopulmonary bypass at a concentration ranging from 30-80 mg/kg body weight. In some embodiments, at least one dose of AAT-1 is administered to a patient having a coronary surgery with a cardiopulmonary bypass at a concentration ranging from 30-70 mg/kg body weight. In some embodiments, at least one dose of AAT-1 is administered to a patient having a coronary surgery with a cardiopulmonary bypass at a concentration ranging from 30-60 mg/kg body weight. In some embodiments, at least one dose of AAT-1 is administered to a patient having a coronary surgery with a cardiopulmonary bypass at a concentration ranging from 30-50 mg/kg body weight. In some embodiments, at least one dose of AAT-1 is administered to a patient having a coronary surgery with a cardiopulmonary bypass at a concentration ranging from 30-40 mg/kg body weight.

In some embodiments, at least one dose of AAT-1 is administered to a patient having a cardiac surgery (e.g., coronary artery bypass surgery) with the use of cardiopulmonary bypass at a concentration ranging from 40-100 mg/kg body weight. In some embodiments, at least one dose of AAT-1 is administered to a patient having cardiac surgery (e.g., coronary artery bypass surgery) with the use of cardiopulmonary bypass at a concentration ranging from 50-100 mg/kg body weight. In some embodiments, at least one dose of AAT-1 is administered to a patient having a cardiac surgery (e.g., coronary artery bypass surgery) with the use of cardiopulmonary bypass at a concentration ranging from 60-100 mg/kg body weight. In some embodiments, at least one dose of AAT-1 is administered to a patient having a cardiac surgery (e.g., coronary artery bypass surgery) with the use of cardiopulmonary bypass at a concentration ranging from 70-100 mg/kg body weight. In some embodiments, at least one dose of AAT-1 is administered to a patient having a cardiac surgery (e.g., coronary artery bypass surgery) with the use of cardiopulmonary bypass at a concentration ranging from 80-100 mg/kg body weight. In some embodiments, at least one dose of AAT-1 is administered to a patient having a cardiac surgery (e.g., coronary artery bypass surgery) with the use of cardiopulmonary bypass at a concentration ranging from 90-100 mg/kg body weight.

In some embodiments, at least one dose of AAT-1 is administered to a patient having a cardiac surgery (e.g., coronary artery bypass surgery) with the use of cardiopulmonary bypass at a concentration ranging from 50-90 mg/kg body weight. In some embodiments, at least one dose of AAT-1 is administered to a patient having a cardiac surgery (e.g., coronary artery bypass surgery) with the use of cardiopulmonary bypass at a concentration ranging from 60-80 mg/kg body weight. In some embodiments, at least one dose of AAT-1 is administered to a patient having a cardiac surgery (e.g., coronary artery bypass surgery) with the use of cardiopulmonary bypass at a concentration ranging from 70-80 mg/kg body weight. In some embodiments, at least one dose of AAT-1 is administered to a patient having a cardiac surgery (e.g., coronary artery bypass surgery) with the use of cardiopulmonary bypass at a concentration ranging from 60-70 mg/kg body weight.

In some embodiments, at least one dose of AAT-1 is administered to a patient having a cardiac surgery (e.g., coronary artery bypass surgery) with the use of cardiopulmonary bypass at a concentration ranging from 40-120 mg/kg body weight. In some embodiments, at least one dose of AAT-1 is administered to a patient having a cardiac surgery (e.g., coronary artery bypass surgery) with the use of cardiopulmonary bypass at a concentration ranging from 50-120 mg/kg body weight. In some embodiments, at least one dose of AAT-1 is administered to a patient having a cardiac surgery (e.g., coronary artery bypass surgery) with the use of cardiopulmonary bypass at a concentration ranging from 60-120 mg/kg body weight. In some embodiments, at least one dose of AAT-1 is administered to a patient having a cardiac surgery (e.g., coronary artery bypass surgery) with the use of cardiopulmonary bypass at a concentration ranging from 70-120 mg/kg body weight. In some embodiments, at least one dose of AAT-1 is administered to a patient having a cardiac surgery (e.g., coronary artery bypass surgery) with the use of cardiopulmonary bypass at a concentration ranging from 80-120 mg/kg body weight. In some embodiments, at least one dose of AAT-1 is administered to a patient having a cardiac surgery (e.g., coronary artery bypass surgery) with the use of cardiopulmonary bypass at a concentration ranging from 90-120 mg/kg body weight. In some embodiments, at least one dose of AAT-1 is administered to a patient having a cardiac surgery (e.g., coronary artery bypass surgery) with the use of cardiopulmonary bypass at a concentration ranging from 100-120 mg/kg body weight. In some embodiments, at least one dose of AAT-1 is administered to a patient having a cardiac surgery (e.g., coronary artery bypass surgery) with the use of cardiopulmonary bypass at a concentration ranging from 110-120 mg/kg body weight.

In some embodiments, at least one dose of AAT-1 is administered to a patient having a cardiac surgery (e.g., coronary artery bypass surgery) with the use of cardiopulmonary bypass at a concentration ranging from 40-110 mg/kg body weight. In some embodiments, at least one dose of AAT-1 is administered to a patient having a cardiac surgery (e.g., coronary artery bypass surgery) with the use of cardiopulmonary bypass at a concentration ranging from 40-100 mg/kg body weight. In some embodiments, at least one dose of AAT-1 is administered to a patient having a cardiac surgery (e.g., coronary artery bypass surgery) with the use of cardiopulmonary bypass at a concentration ranging from 40-90 mg/kg body weight. In some embodiments, at least one dose of AAT-1 is administered to a patient having a cardiac surgery (e.g., coronary artery bypass surgery) with the use of cardiopulmonary bypass at a concentration ranging from 40-80 mg/kg body weight. In some embodiments, at least one dose of AAT-1 is administered to a patient having a cardiac surgery (e.g., coronary artery bypass surgery) with the use of cardiopulmonary bypass at a concentration ranging from 40-70 mg/kg body weight. In some embodiments, at least one dose of AAT-1 is administered to a patient having a cardiac surgery (e.g., coronary artery bypass surgery) with the use of cardiopulmonary bypass at a concentration ranging from 40-60 mg/kg body weight. In some embodiments, at least one dose of AAT-1 is administered to a patient having a cardiac surgery (e.g., coronary artery bypass surgery) with the use of cardiopulmonary bypass at a concentration ranging from 40-50 mg/kg body weight.

In some embodiments, at least one dose of AAT-1 is administered to a patient having a cardiac surgery (e.g., coronary artery bypass surgery) with the use of cardiopulmonary bypass at a concentration ranging from 50-110 mg/kg body weight. In some embodiments, at least one dose of AAT-1 is administered to a patient having a cardiac surgery (e.g., coronary artery bypass surgery) with the use of cardiopulmonary bypass at a concentration ranging from 60-100 mg/kg body weight. In some embodiments, at least one dose of AAT-1 is administered to a patient having a cardiac surgery (e.g., coronary artery bypass surgery) with the use of cardiopulmonary bypass at a concentration ranging from 70-90 mg/kg body weight. In some embodiments, at least one dose of AAT-1 is administered to a patient having a cardiac surgery (e.g., coronary artery bypass surgery) with the use of cardiopulmonary bypass at a concentration ranging from 70-80 mg/kg body weight. In some embodiments, at least one dose of AAT-1 is administered to a patient having a cardiac surgery (e.g., coronary artery bypass surgery) with the use of cardiopulmonary bypass at a concentration ranging from 80-90 mg/kg body weight.

In some embodiments, administration of AAT-1 to a patient having a cardiac surgery (e.g., a coronary artery bypass surgery) with the use of cardiopulmonary bypass results in 4-fold increase in AAT-1 plasma levels, where this increase in AAT-1 plasma levels is similar to (e.g., 75%-125%) a normal response to an inflammatory stimulus. In some embodiments, administering fluids to a patient having a cardiac surgery (e.g., a coronary artery bypass surgery) with the use of cardiopulmonary bypass can result in a dilution effect. In some embodiments, the dilution effect of the administered fluids during the procedure (e.g., approximately 1.5 liters administered for CPB machine priming and 1 liter administered intravenous during induction of anesthesia) results in a 1.5-fold increase in the effective blood volume compared to preoperative values.

In some embodiments, the composition is administered as a single dose. In other embodiments, the composition is administered in multiple doses.

In some embodiments, the sole active ingredient of the composition is AAT-1. In other embodiments, the composition includes multiple active ingredients, and particularly at least one additional active ingredient for treatment of injury resultant from cardiac surgery. In still other embodiments, the additional one or more active ingredients is administered to the subject in an additional composition, which can be administered to the subject prior to, concurrent with, or after administration of the composition comprising AAT-1.

In some embodiments the subject is human. In other embodiments, the subject is a veterinary subject.

Also described herein are methods for treating or preventing injury during or resultant from cardiac surgery in a human or veterinary subject, by administering to the subject a composition comprising a therapeutically effective amount AAT-1, or a functional variant thereof.

Some embodiments of the described methods are directed to treatment or prevention of the injury resulting from use of cardiac bypass, including excessive post-operative bleeding or organ injury.

In some embodiments, the composition is administered to the subject before the cardiac surgery, during the cardiac surgery, after the cardiac surgery or a combination thereof.

In some embodiments, the concentration of AAT-1, or functional variant thereof, in the composition is 1 gram in 50 cc sterile fluid in the form of a sterile or physiologically isotonic aqueous solution.

In some embodiments, the AAT-1, or functional variant thereof, is administered to the subject at a concentration of 30 to 300 mg per kg body weight per day. In other embodiments, the AAT-1 is administered to the subject at a concentration of 60 to 120 mg per kg body weight per day. In other embodiments, the AAT-1 is administered to the subject at a concentration of 60 to 100 mg per kg body weight per day.

In some embodiments, the AAT-1-containing composition is administered as a single dose. In other embodiments, the AAT-1-containing composition is administered in multiple doses.

In some embodiments, of the described methods, the composition comprises at least one additional composition for treatment of injury resultant from cardiac surgery. In other embodiments, the methods comprise administration of at least one additional composition which contains one or more active ingredients for treatment of injury resultant from cardiac surgery, which is administered to the subject prior to, concurrent with, or after administration of the composition comprising AAT-1.

### IV. Cardiopulmonary Bypass

Cardiopulmonary bypass (CPB) during cardiac surgery elicits generalized non-specific systemic inflammatory response syndrome (SIRS), which initiates the activation of cytokine, complement, and coagulation-fibrinolytic cascades. In approximately 1% of all patients, depending on the number of organs involved, SIRS may result in severe multi-organ failure (MOF), having a mortality rate of 40-98%. Strategies used to attenuate the effects of SIRS focus on optimization of anaesthesiological, surgical, and CPB techniques.

During CPB, passage of blood through plastic tubing and through an oxygenator activates the clotting cascade, including activation of complement, cytokines, platelets, neutrophils, adhesion molecules, mast cells, and multiple inflammatory mediators. This can generate multi-organ system dysfunction that can manifest in a subject as post-operative respiratory failure, myocardial dysfunction, renal insufficiency, and neurocognitive defects.

The mechanism of damage to the lungs during cardiopulmonary bypass is unique. During CBP, blood flow through pulmonary circulation is minimal. This is followed by sequestration of neutrophils in the pulmonary capillary bed probably secondary to the lack of blood flow and the activation of pro inflammatory cytokines. The sequestered neutrophils, through the release of proteolytic enzymes, cause endothelial cell swelling, plasma and protein extravasation into the interstitial tissue, congestion of the alveoli with plasma, erythrocytes and inflammatory debris. Coagulation and inflammation are closely linked during cardiopulmonary bypass through networks of both humoral and cellular components including activation of proteases of the clotting and fibrinolytic cascades.

### V. Methods for Inhibiting or Preventing Injury During or Resultant from Cardiac Surgery

Described herein are methods of treating or preventing injury to a subject associated with cardiac surgery. The described methods involve administration of a composition comprising an effective amount of at least one dose of alpha 1 antitrypsin (AAT-1) prior to, concurrent with, or following cardiac surgery.

In some embodiments the AAT-1-containing composition can be administered to the subject prior to the start of cardiac surgery such as 1, 2, 3, 4, 5 or more hours before surgery, including prior to administration of anesthesia or during pre-operative preparations. In other embodiments, the AAT-1-containing composition can be administered during cardiac surgery. In still other embodiments, the AAT-1 containing composition can be administered following the surgery, such as 1, 2, 3, 4, 5 or more hours after surgery, or 1, 2, 3, 4, 5 or more days following surgery

In some embodiments, the cardiac surgery involves use of cardiopulmonary bypass. In such embodiments, the at least one dose of AAT-1 is administered to the subject prior, current with, or after use of a cardiopulmonary bypass machine, but while the surgery is ongoing.

Cardiac surgery employing CPB can result in particular physiological pathologies in a subject, such as excessive post-operative bleeding and/or organ damage. The compositions and methods described herein can treat such pathologies, and therefore decrease the severity of the post-operative bleeding and/or organ damage. In particular examples, administration of an AAT-1 containing composition to a subject prior to, during, or following cardiac surgery employing CPB can prevent the injury. In such examples, post-operative bleeding is prevented from occurring as is organ damage. The development of post-operative bleeding and organ damage is determined by standard methods known to the art.

### Alpha-1 Antitrypsin (AAT-1)

AAT-1 is a plasma-derived protein belonging to the family of serine proteinase inhibitors. AAT-1 is synthesized primarily in the liver, and to a lesser extent in other cells, including macrophages, intestinal epithelial cells and intestinal Paneth cells. In the liver, AAT-1 is initially synthesized as a 52 kD precursor protein that subsequently undergoes post translational glycosylation at three asparagine residues, as well as tyrosine sulfonation. The resulting mature protein is secreted as a 55 kD native single-chain glycoprotein. AAT-1 is also known as SERPINA-1. Nucleotide and amino acid sequences of human AAT-1 are available on-line at ncbi.nlm.nih.gov/nuccore/189163524 and ncbi.nlm.nih.gov/protein/NP 000286, respectively, and are included herein as SEQ ID NOs: 1 and 2.

AAT-1 is associated with control of tissue destruction by endogenous serine proteinases, and is the most prevalent serine proteinase inhibitor in blood plasma. AAT-1 inhibits, inter alia, trypsin, chymotrypsin, various types of elastases, skin collagenase, renin, urokinase and proteases of polymorphonuclear lymphocytes.

Patients with low circulating levels of AAT-1 are at increased risk for lung, liver, and pancreatic diseases, particularly emphysema. Accumulating data suggests that besides its ability to inhibit serine proteases, AAT-1 possesses independent anti-inflammatory and tissue-protective effects. AAT-1 modifies dendritic cell maturation and promotes regulatory T-cell differentiation, induces interleukin (IL)-1 receptor antagonist and IL-10 release, protects various cell types from cell death, inhibits caspase-1 and caspase-3 activity and inhibits IL-1 production and activity.

Importantly, and contradictory to classic immune-suppressants, AAT-1 allows undeterred isolated T-lymphocyte responses. AAT-1 is currently used therapeutically for the treatment of pulmonary emphysema in AAT-1-deficient patients. AAT-1 deficiency is a genetic condition that increases the risk of developing a variety of diseases including pulmonary emphysema. AAT-1 deficiency is a result of mutations in the AAT-1-encoding gene (proteinase inhibitor (Pi) gene). Purified AAT-1 has been approved for replacement therapy (also known as “augmentation therapy”) in such patients deficient in endogenous AAT-1.

AAT-1 is currently administered intravenously, and commercially-available AAT-1 preparations can be used in the methods and compositions described herein. For example, AAT-1 marketed under tradenames including Aralast® (Baxter Healthcare Corporation, Westlake Village, Calif.); Zemaira® (CSL Behring, King of Prussia, Pa.); Prolastin® (Grifols Therapeutics Inc., Clayton, N.C.); Trypsone® (Evaluate, Ltd); and Alfalastin®, and which are human AAT-1 formulations indicated for augmentation therapy in patients congenital deficiency of AAT-1 with clinically evident emphysema, can be used as described herein.

In addition to the commercially-available preparations, compositions comprising AAT-1 can be produced by standard protein expression and purification methodology known to the art and formulated for administration as described herein. It is also appreciated that functional variants of AAT-1 can be produced by standard methods of mutagenesis, which will maintain the activity of the wild type protein, and can be used in the compositions and methods described herein. Such functional variants can be identical in sequence to wild type AAT-1 by at least 99%, at least 98%, at least 95%, at least 90%, at least 85%, at least 80%, or even less than 80% identical (e.g., between 50%, 55%, 60%, 65%, 70%, 75%, etc.).

### Combination Therapies

In some embodiments of the compositions and methods described herein, AAT-1 is combined with at least one additional active agent to treat or prevent injury resultant from excessive post-operative bleeding and/or organ damage. Non-limiting examples of active compounds for decreasing post-operative bleeding include fresh frozen plasma, platelets, cryoprecipitate, and alpha aminocaproic acid. Non-limiting examples of active compounds and procedures that can be used to decrease organ damage include steroids, and leucocyte depletion methods.

In some embodiments, the combination of AAT-1 and at least one additional active agent is administered to a subject in a single composition. In particular examples, the combination compositions are formulated so that the component active ingredients are simultaneously available in the subject in an active form. In other examples, the component active ingredients are formulated such that the components are sequentially available in an active form to the subject. For example, although administered simultaneously, the AAT-1 might produce the desired effect prior to the at least one additional compound.

In other embodiments, combinations of AAT-1 and at least one additional active agent can be administered to a subject in multiple compositions, one containing AAT-1 and at least one additional composition containing the at least one additional active agent. The timing and order of administration of such multiple compositions can vary, such as prior to, during, and after cardiac surgery, as described herein. In particular examples, the AAT-1-containing composition is administered prior to the additional composition. In other examples, the AAT-1-containing composition is administered simultaneously with the additional composition. In still other embodiments, the AAT-1-containing composition is administered after the additional composition. It is contemplated that when administered at separate times, significant time may elapse between administration of the at least two compositions, such as several hours, several days or even longer.

### Pharmaceutical Compositions and Modes of Administration

The AAT-1 and other active agents for use in the described compositions and methods can be supplied in any pharmaceutically acceptable compositions. As described herein, AAT-1 is currently commercially available in several intravenous formulations.

Additionally, the pharmaceutical compositions specifically contemplated in the present disclosure can include pharmaceutically acceptable acid or base addition salts. The phrase “pharmaceutically acceptable acid or base addition salts” includes therapeutically active non-toxic acid and non-toxic base addition salt forms which at least some of the active agents described herein can form. Such compounds which have basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

Those active agents which have acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

Various delivery systems are known and can be used to administer peptide-based (such as AAT-1) and non-peptide active agents as therapeutics. Such systems include, for example, encapsulation in liposomes, microparticles, microcapsules, recombinant cells capable of expressing therapeutic molecule(s), construction of a therapeutic nucleic acid as part of a retroviral or other vector, and the like. Although current AAT-1 formulations are administered to subject intravenously, various alternative methods of administration of AAT-1 or additional active agents include, but are not limited to, intrathecal, intradermal, intramuscular, intraperitoneal (ip), intravenous (iv), subcutaneous, intranasal, epidural, and oral routes. The active agent therapeutics may be administered by any convenient route, including, for example, infusion or bolus injection, topical, absorption through epithelial or mucocutaneous linings (e.g., oral mucosa, rectal and intestinal mucosa, and the like) ophthalmic, nasal, and transdermal, and may be administered together with other biologically active agents. Pulmonary administration can also be employed (e.g., by an inhaler or nebulizer), for instance using a formulation containing an aerosolizing agent.

In a specific embodiment, it may be desirable to administer the described compositions by injection, catheter, suppository, or implant (e.g., implants formed from porous, non-porous, or gelatinous materials, including membranes, such as sialastic membranes or fibers), and the like. In another embodiment, therapeutic agents are delivered in a vesicle, in particular liposomes.

In yet another embodiment, any one of the agents described herein can be delivered in a controlled release system. In one embodiment, a pump may be used. In another embodiment, polymeric materials and/or any other controlled release system can be used.

As described above, in particular examples wherein AAT-1 is administered with at least one additional active agent, the active agents are administered simultaneously, and by the same mode of administration. In other examples, the pharmaceutical compounds are administered at different times, and either by the same or different more of administration.

The vehicle in which the agent is delivered can include pharmaceutically acceptable compositions of the compounds, using methods well known to those with skill in the art. For instance, in some embodiments, described active agents typically are contained in a pharmaceutically acceptable carrier. The term “pharmaceutically acceptable” means approved by a regulatory agency of the federal or a state government or listed in the U.S. Pharmacopoeia or other generally recognized pharmacopoeia for use in animals, and, more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable, or synthetic origin, such as peanut oil, soybean oil, mineral oil, sesame oil, and the like. Water is a preferred carrier when the pharmaceutical composition is administered intravenously. Saline solutions, blood plasma medium, aqueous dextrose, and glycerol solutions can also be employed as liquid carriers, particularly for injectable solutions. The medium may also contain conventional pharmaceutical adjunct materials such as, for example, pharmaceutically acceptable salts to adjust the osmotic pressure, lipid carriers such as cyclodextrins, proteins such as serum albumin, hydrophilic agents such as methyl cellulose, detergents, buffers, preservatives and the like.

Examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The therapeutic, if desired, can also contain minor amounts of wetting or emulsifying agents, or pH buffering agents. The therapeutics can take the form of solutions, suspensions, emulsion, tablets, pills, capsules, powders, sustained-release formulations, and the like. The therapeutic can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like.

Embodiments of other pharmaceutical compositions are prepared with conventional pharmaceutically acceptable counter-ions, as would be known to those of skill in the art.

Therapeutic preparations will contain a therapeutically effective amount of at least one active ingredient, preferably in purified form, together with a suitable amount of carrier so as to provide proper administration to the patient. The formulation should suit the mode of administration.

The compositions of this disclosure can be formulated in accordance with routine procedures as pharmaceutical compositions adapted for intravenous administration to human beings. Typically, compositions for intravenous administration are solutions in sterile isotonic aqueous buffer. Where necessary, the compositions may also include a solubilizing agent and a local anesthetic such as lidocaine to ease pain at the site of the injection.

The ingredients in various embodiments are supplied either separately or mixed together in unit dosage form, for example, in solid, semi-solid and liquid dosage forms such as tablets, pills, powders, liquid solutions, or suspensions, or as a dry lyophilized powder or water free concentrate in a hermetically sealed container such as an ampoule or sachette indicating the quantity of active agent. Where one or more of the indicated agents is to be administered by infusion, it can be dispensed with an infusion bottle containing sterile pharmaceutical grade water or saline. Where one or more of the indicated agents is to be administered by injection, an ampoule of sterile water or saline can be provided so that the ingredients may be mixed prior to administration.

Effective amounts can be determined by standard clinical techniques. The precise dose to be employed in the formulation will also depend on the route of administration, and should be decided according to the judgment of the health care practitioner and each patient's circumstances. Exemplary dosages of the individual compounds are described herein, but myriad other dosage regimens are encompassed by this disclosure. An example of an additional dosage range is 0.1 to 200 mg/kg body weight in single or divided doses (e.g., but not limited to, two doses, three doses, etc.). Another example of a dosage range is 1.0 to 100 mg/kg body weight in single or divided doses (e.g., but not limited to, two doses, three doses, etc.).

In some embodiments, AAT-1 is provided in a composition at a concentration of 1 gram in 50 cc sterile fluid in the form of a sterile or physiologically isotonic aqueous solution. In some embodiments a single dosage of AAT-1 administered to a subject at a dosage of 30 mg to 400 mg per kg body weight per day, such as, but not limited to, 60 mg to 100 mg per kg body weight per day. In some embodiments, multiple comparable dosages of AAT-1 are administered to a subject in a combination of dosing periods prior to, during, or after cardiac surgery.

The specific dose level and frequency of dosage for any particular subject may be varied and will depend upon a variety of factors, including the activity of the specific compound, the metabolic stability and length of action of that compound, the age, body weight, general health, sex, diet, mode and time of administration, rate of excretion, drug combination, and severity of the condition of the host undergoing therapy.

In some embodiments, sustained localized release of the pharmaceutical preparation that comprises a therapeutically effective amount of a therapeutic compound or composition may be beneficial. Slow-release formulations are known to those of ordinary skill in the art. By way of example, polymers such as bis(p-carboxyphenoxy)propane-sebacic-acid or lecithin suspensions may be used to provide sustained localized release.

In some embodiments of the inventive methods of the present invention, a reduction in bleeding/blood loss can result from administering AAT-1 to a patient having a coronary surgery with a cardiopulmonary bypass. In some embodiments, operative and postoperative bleeding/blood loss was monitored using hourly chest drainage measurement. In some embodiments, the distribution of blood products and total blood administered to the patient was recorded daily. In some embodiments, the reduction in bleeding/blood loss measures at least 30% compared with a patient having a coronary surgery with a cardiopulmonary bypass and not administered AAT-1. In some embodiments, the reduction in bleeding/blood loss measures at least 30% compared with a patient having a coronary surgery with a cardiopulmonary bypass and not administered AAT-1. In some embodiments, the reduction in consumption of blood products is directly proportional to the reduction in bleeding/blood loss of a patient (e.g., 30% less blood loss results in a patient being administered 30% less blood products. In some embodiments, a blood product is, but is not limited to, whole blood (e.g., donated blood), packed cells, fresh frozen plasma, cryoprecipitate, thrombocytes, or any combination thereof.

In some embodiments, the reduction in bleeding/blood loss is between 30% and 60% compared with a patient having a coronary surgery with a cardiopulmonary bypass and not administered AAT-1. In some embodiments, the reduction in bleeding/blood loss measures between 30% and 50% compared with a patient having a coronary surgery with a cardiopulmonary bypass and not administered AAT-1. In some embodiments, the reduction in bleeding/blood loss measures between 30% and 45% compared with a patient having a coronary surgery with a cardiopulmonary bypass and not administered AAT-1. In some embodiments, the reduction in bleeding/blood loss measures between 30% and 40% compared with a patient having a coronary surgery with a cardiopulmonary bypass and not administered AAT-1. In some embodiments, the reduction in bleeding/blood loss measures between 30% and 35% compared with a patient having a coronary surgery with a cardiopulmonary bypass and not administered AAT-1. In some embodiments, the reduction in bleeding/blood loss measures between 35% and 60% compared with a patient having a coronary surgery with a cardiopulmonary bypass and not administered AAT-1. In some embodiments, the reduction in bleeding/blood loss measures between 40% and 60% compared with a patient having a coronary surgery with a cardiopulmonary bypass and not administered AAT-1. In some embodiments, the reduction in bleeding/blood loss measures between 45% and 60% compared with a patient having a coronary surgery with a cardiopulmonary bypass and not administered AAT-1. In some embodiments, the reduction in bleeding/blood loss measures between 50% and 60% compared with a patient having a coronary surgery with a cardiopulmonary bypass and not administered AAT-1.

In some embodiments, the reduction in bleeding/blood loss measures between 35% and 55% compared with a patient having a coronary surgery with a cardiopulmonary bypass and not administered AAT-1. In some embodiments, the reduction in bleeding/blood loss measures between 40% and 50% compared with a patient having a coronary surgery with a cardiopulmonary bypass and not administered AAT-1.

In some embodiments of the inventive methods of the present invention, a reduction in BBB disruption can result from administering AAT-1 to a patient having a coronary surgery with a cardiopulmonary bypass. In some embodiments, the reduction in BBB disruption measures at least 30% compared with a patient having a coronary surgery with a cardiopulmonary bypass and not administered AAT-1. In some embodiments, the reduction in BBB disruption measures between 30% and 60% compared with a patient having a coronary surgery with a cardiopulmonary bypass and not administered AAT-1. In some embodiments, the reduction in BBB disruption measures between 30% and 50% compared with a patient having a coronary surgery with a cardiopulmonary bypass and not administered AAT-1. In some embodiments, the reduction in BBB disruption measures between 30% and 45% compared with a patient having a coronary surgery with a cardiopulmonary bypass and not administered AAT-1. In some embodiments, the reduction in BBB disruption measures between 30% and 40% compared with a patient having a coronary surgery with a cardiopulmonary bypass and not administered AAT-1. In some embodiments, the reduction in BBB disruption measures between 30% and 35% compared with a patient having a coronary surgery with a cardiopulmonary bypass and not administered AAT-1. In some embodiments, the reduction in BBB disruption is between 35% and 60% compared with a patient having a coronary surgery with a cardiopulmonary bypass and not administered AAT-1. In some embodiments, the reduction in BBB disruption measures between 40% and 60% compared with a patient having a coronary surgery with a cardiopulmonary bypass and not administered AAT-1. In some embodiments, the reduction in BBB disruption measures between 45% and 60% compared with a patient having a coronary surgery with a cardiopulmonary bypass and not administered AAT-1. In some embodiments, the reduction in BBB disruption measures between 50% and 60% compared with a patient having a coronary surgery with a cardiopulmonary bypass and not administered AAT-1.

In some embodiments, the reduction in BBB disruption measures between 35% and 55% compared with a patient having a coronary surgery with a cardiopulmonary bypass and not administered AAT-1. In some embodiments, the reduction in BBB disruption measures between 40% and 50% compared with a patient having a coronary surgery with a cardiopulmonary bypass and not administered AAT-1.

In some embodiments of the inventive methods of the present invention, a reduction in levels of postoperative inflammatory markers can result from administering AAT-1 to a patient having a coronary surgery with a cardiopulmonary bypass. In some embodiments, the reduction in levels of postoperative inflammatory markers measures at least 30% compared with a patient having a coronary surgery with a cardiopulmonary bypass and not administered AAT-1. In some embodiments, the reduction in levels of postoperative inflammatory markers is between 30% and 60%. In some embodiments, the reduction in levels of postoperative inflammatory markers measures between 30% and 50% compared with a patient having a coronary surgery with a cardiopulmonary bypass and not administered AAT-1. In some embodiments, the reduction in levels of postoperative inflammatory markers measures between 30% and 45% compared with a patient having a coronary surgery with a cardiopulmonary bypass and not administered AAT-1. In some embodiments, the reduction in levels of postoperative inflammatory markers measures between 30% and 40% compared with a patient having a coronary surgery with a cardiopulmonary bypass and not administered AAT-1. In some embodiments, the reduction in levels of postoperative inflammatory markers measures between 30% and 35% compared with a patient having a coronary surgery with a cardiopulmonary bypass and not administered AAT-1. In some embodiments, the reduction in levels of postoperative inflammatory markers measures between 35% and 60% compared with a patient having a coronary surgery with a cardiopulmonary bypass and not administered AAT-1. In some embodiments, the reduction in levels of postoperative inflammatory markers measures between 40% and 60% compared with a patient having a coronary surgery with a cardiopulmonary bypass and not administered AAT-1. In some embodiments, the reduction in levels of postoperative inflammatory markers measures between 45% and 60% compared with a patient having a coronary surgery with a cardiopulmonary bypass and not administered AAT-1. In some embodiments, the reduction in levels of postoperative inflammatory markers measures between 50% and 60% compared with a patient having a coronary surgery with a cardiopulmonary bypass and not administered AAT-1.

In some embodiments, the reduction in levels of postoperative inflammatory markers measures between 35% and 55% compared with a patient having a coronary surgery with a cardiopulmonary bypass and not administered AAT-1. In some embodiments, the reduction in levels of postoperative inflammatory markers measures between 40% and 50% compared with a patient having a coronary surgery with a cardiopulmonary bypass and not administered AAT-1.

In some embodiments of the inventive methods of the present invention, a reduced AaDO₂ measurement can result from administering AAT-1 to a patient having a coronary surgery with a cardiopulmonary bypass. In some embodiments, the reduced AaDO₂ measurement is reduced by at least 30% compared with a patient having a coronary surgery with a cardiopulmonary bypass and not administered AAT-1. In some embodiments, the reduced AaDO₂ measurement is between 30% and 60% compared with a patient having a coronary surgery with a cardiopulmonary bypass and not administered AAT-1. In some embodiments, the reduced AaDO₂ measurement is between 30% and 50% compared with a patient having a coronary surgery with a cardiopulmonary bypass and not administered AAT-1. In some embodiments, the reduction in BBB disruption is between 30% and 45% compared with a patient having a coronary surgery with a cardiopulmonary bypass and not administered AAT-1. In some embodiments, the reduced AaDO₂ measurement is between 30% and 40% compared with a patient having a coronary surgery with a cardiopulmonary bypass and not administered AAT-1. In some embodiments, the reduced AaDO₂ measurement is between 30% and 35% compared with a patient having a coronary surgery with a cardiopulmonary bypass and not administered AAT-1. In some embodiments, the reduced AaDO₂ measurement is between 35% and 60% compared with a patient having a coronary surgery with a cardiopulmonary bypass and not administered AAT-1. In some embodiments, the reduced AaDO₂ measurement is between 40% and 60% compared with a patient having a coronary surgery with a cardiopulmonary bypass and not administered AAT-1. In some embodiments, the reduced AaDO₂ measurement is between 45% and 60% compared with a patient having a coronary surgery with a cardiopulmonary bypass and not administered AAT-1. In some embodiments, the reduced AaDO₂ measurement is between 50% and 60% compared with a patient having a coronary surgery with a cardiopulmonary bypass and not administered AAT-1.

In some embodiments, the reduced AaDO₂ measurement is between 35% and 55% compared with a patient having a coronary surgery with a cardiopulmonary bypass and not administered AAT-1. In some embodiments, the reduced AaDO₂ measurement is between 40% and 50% compared with a patient having a coronary surgery with a cardiopulmonary bypass and not administered AAT-1.

It is specifically contemplated in some embodiments that delivery is via an injected and/or implanted drug depot, for instance comprising multi-vesicular liposomes (e.g., DepoFoam (SkyePharma, Inc, San Diego, Calif.).

In some embodiments, a reduced CPB-inflicted inflammatory reaction can mean a reduction in levels of postoperative inflammatory markers (e.g., IL-6, TNF-alpha, IL-1 beta, IL-8, MCP-1, LDH, endotoxin, C3a, kinin, kalikrein, soluble adhesion molecules (e.g., sICAM-1, sVCAM-1, sE-selectin and sP-selectin), metalloproteinase, elastase, nucleic factor kb, D dimmer, or any combination thereof) and can be measured using specific ELISA kits (e.g., but not limited to the following: IL-6 can be measured by use of Life Technologies Kit Catalog Number KHC0061; TNF alpha can be measured by use of Life Technologies Kit Catalog NumberKHC3011; IL-1 beta can be measured by use of Life Technologies Kit Catalog NumberKHC0011; IL-8 can be measured by use of Life Technologies Kit Catalog NumberKHC0081; MCP-1 can be measured by use of Life Technologies Kit Catalog NumberKHC1011; endotoxin can be measured by use of Hyglos GmbH EndoLISA; C3a can be measured by use of Enzo Life Sciences Kit ADI 900 058; Kinin can be measured by use of Enzo Life Sciences Kit ADI-900-206; kalikrein can be measured by use of Enzo Life Sciences Kit ADI-900-218-0001; Adhesion molecules can be measured by use of Biotrend Chemikalien GmBh Kit Catalog Number E0216Hu-48; Metalloproteinase can be measured by use of Biosensis Kit Catalogue Number BEK-2067-2P, elastase can be measured by use of Abcam Kit ab119553, Nucleic Factor kb can be measured by use of Active Motif Kit Catalogue Number 43296, D Dimer can be measured by use of Abbexa Kit Catalogue No. abx51360) and result in a 3-fold reduction of a CPB-inflicted inflammatory reaction. In some embodiments, a reduced CPB-inflicted inflammatory reaction can mean a reduction in levels of postoperative inflammatory markers (e.g., IL-6, TNF-alpha, IL-1 beta, IL-8, MCP-1, LDH, endotoxin, C3a, kinin, kalikrein, soluble adhesion molecules (e.g., sICAM-1, sVCAM-1, sE-selectin and sP-selectin), metalloproteinase, elastase, nucleic factor kb, D dimmer, or any combination thereof) and result in a 2-4-fold reduction of a CPB-inflicted inflammatory reaction. In some embodiments, a reduced CPB-inflicted inflammatory reaction can mean a reduction in levels of postoperative inflammatory markers (e.g., IL-6, TNF-alpha, IL-1 beta, IL-8, MCP-1, LDH, endotoxin, C3a, kinin, kalikrein, soluble adhesion molecules (e.g., sICAM-1, sVCAM-1, sE-selectin and sP-selectin), metalloproteinase, elastase, nucleic factor kb, D dimmer, or any combination thereof) and result in a 3-4-fold reduction of a CPB-inflicted inflammatory reaction. In some embodiments, a reduced CPB-inflicted inflammatory reaction can mean a reduction in levels of postoperative inflammatory markers (e.g., IL-6, TNF-alpha, IL-1 beta, IL-8, MCP-1, LDH, endotoxin, C3a, kinin, kalikrein, soluble adhesion molecules (e.g., sICAM-1, sVCAM-1, sE-selectin and sP-selectin), metalloproteinase, elastase, nucleic factor kb, D dimmer, or any combination thereof) and result in a 2-3-fold reduction of a CPB-inflicted inflammatory reaction.

In some embodiments, a reduced CPB-inflicted organ injury can mean an increase in postoperative levels of anti-inflammatory cytokine markers (e.g., IL-1 receptor antagonist and/or IL-10) and can be measured by at least one ELISA kit (e.g., but not limited to: IL-1 can be measured by use of Abcam Kit ab100565; IL-10 can be measured by use of R and D Systems Kit catalogue no. D1000B) and result in a 40% increase of anti-inflammatory cytokine markers. In some embodiments, a reduced CPB-inflicted organ injury can mean an increase in postoperative levels of anti-inflammatory cytokine markers (e.g., IL-1 receptor antagonist and/or IL-10) and can be measured by at least one ELISA kit (e.g., but not limited to: IL-1 can be measured by use of Abcam Kit ab100565; IL-10 can be measured by use of R and D Systems Kit catalogue no. D1000B) and result in a 30-50% increase of anti-inflammatory cytokine markers. In some embodiments, a reduced CPB-inflicted organ injury can mean an increase in postoperative levels of anti-inflammatory cytokine markers (e.g., IL-1 receptor antagonist and/or IL-10) and can be measured by at least one ELISA kit (e.g., but not limited to: IL-1 can be measured by use of Abcam Kit ab100565; IL-10 can be measured by use of R and D Systems Kit catalogue no. D1000B) and result in a 30-40% increase of anti-inflammatory cytokine markers. In some embodiments, a reduced CPB-inflicted organ injury can mean an increase in postoperative levels of anti-inflammatory cytokine markers (e.g., IL-1 receptor antagonist and/or IL-10) and can be measured by at least one ELISA kit (e.g., but not limited to: IL-1 can be measured by use of Abcam Kit ab100565; IL-10 can be measured by use of R and D Systems Kit catalogue no. D1000B) and result in a 40-50% increase of anti-inflammatory cytokine markers.

In some embodiments, a reduced CPB-inflicted organ injury can mean a reduction of elevation in blood liver enzymes and can be measured by levels of glutamic oxaloacetic transaminase (GOT) and/or glutamic pyruvic transaminase (GPT) by measuring serum enzyme activity, and result in a 40% reduction of a CPB-inflicted elevated serum enzyme activity. In some embodiments, a reduced CPB-inflicted organ injury can mean a reduction of elevation in blood liver enzymes and can be measured by levels of glutamic oxaloacetic transaminase (GOT) and/or glutamic pyruvic transaminase (GPT) by measuring serum enzyme activity, and result in a 30-50% reduction of a CPB-inflicted elevated serum enzyme activity. In some embodiments, a reduced CPB-inflicted organ injury can mean a reduction of elevation in blood liver enzymes and can be measured by levels of glutamic oxaloacetic transaminase (GOT) and/or glutamic pyruvic transaminase (GPT) by measuring serum enzyme activity, and result in a 40-50% reduction of a CPB-inflicted elevated serum enzyme activity. In some embodiments, a reduced CPB-inflicted organ injury can mean a reduction of elevation in blood liver enzymes and can be measured by levels of glutamic oxaloacetic transaminase (GOT) and/or glutamic pyruvic transaminase (GPT) by measuring serum enzyme activity, and result in a 30-40% reduction of a CPB-inflicted elevated serum enzyme activity.

In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in a 30% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 15-45% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 20-45% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 25-45% reduction of a

CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 30-45% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 35-45% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 40-45% reduction of a

CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 15-40% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 15-35% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 15-30% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 15-25% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 15-20% reduction of a CPB-inflicted AKI markers elevation.

In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 20-40% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 25-35% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 20-30% reduction of a CPB-inflicted AKI markers elevation. In some embodiments, a reduced CPB-inflicted organ injury can mean a reduction of elevation of renal enzymes and can be measured by AKI markers (e.g., KIM-1, cystatin, N-Gal, or any combination thereof) by specific ELISA kits and result in between 30-40% reduction of a CPB-inflicted AKI markers elevation.

In some embodiments, a reduced CPB-inflicted organ injury can mean a reduction of postoperative bleeding/blood loss and can be measured by quantifying the blood drained from the chest and result in a 40% reduction of a CPB-inflicted bleeding tendency. In some embodiments, a reduced CPB-inflicted organ injury can mean a reduction of postoperative bleeding/blood loss and can be measured by quantifying the blood drained from the chest and result in a 30-50% reduction of a CPB-inflicted bleeding tendency. In some embodiments, a reduced CPB-inflicted organ injury can mean a reduction of postoperative bleeding/blood loss and can be measured by quantifying the blood drained from the chest and result in a 40-50% reduction of a CPB-inflicted bleeding tendency. In some embodiments, a reduced CPB-inflicted organ injury can mean a reduction of postoperative bleeding/blood loss and can be measured by quantifying the blood drained from the chest and result in a 30-40% reduction of a CPB-inflicted bleeding tendency.

In some embodiments, a reduced CPB-inflicted organ injury can mean a reduction of postoperative thrombocytes function and can be measured by thromboelastography (i.e., typical method(s) of performing and/or measuring thromboelastography) and result in a 40% reduction of a CPB-inflicted thrombocytes dysfunction. In some embodiments, a reduced CPB-inflicted organ injury can mean a reduction of postoperative thrombocytes function and can be measured by thromboelastography (i.e., typical method(s) of performing and/or measuring thromboelastography) and result in a 30-50% reduction of a CPB-inflicted thrombocytes dysfunction. In some embodiments, a reduced CPB-inflicted organ injury can mean a reduction of postoperative thrombocytes function and can be measured by thromboelastography (i.e., typical method(s) of performing and/or measuring thromboelastography) and result in a 40-50% reduction of a CPB-inflicted thrombocytes dysfunction. In some embodiments, a reduced CPB-inflicted organ injury can mean a reduction of postoperative thrombocytes function and can be measured by thromboelastography (i.e., typical method(s) of performing and/or measuring thromboelastography) and result in a 30-40% reduction of a CPB-inflicted thrombocytes dysfunction.

In some embodiments, a reduced CPB-inflicted organ injury can mean a reduction of BBB disruption and can be measured by MRI−BBB disruption detection (i.e., typical MRI−BBB disruption detection method(s)) and result in a 40% reduction of a CPB-inflicted BBB disruption incidence. In some embodiments, a reduced CPB-inflicted organ injury can mean a reduction of BBB disruption and can be measured by MRI−BBB disruption detection (i.e., typical MRI−BBB disruption detection method(s)) and result in a 30-50% reduction of a CPB-inflicted BBB disruption incidence. In some embodiments, a reduced CPB-inflicted organ injury can mean a reduction of BBB disruption and can be measured by MRI−BBB disruption detection (i.e., typical MRI−BBB disruption detection method(s)) and result in a 40-50% reduction of a CPB-inflicted BBB disruption incidence. In some embodiments, a reduced CPB-inflicted organ injury can mean a reduction of BBB disruption and can be measured by MRI−BBB disruption detection (i.e., typical MRI−BBB disruption detection method(s)) and result in a 30-40% reduction of a CPB-inflicted BBB disruption incidence.

In some embodiments, a reduced CPB-inflicted organ injury can mean a reduction of BBB disruption and can be measured by quantitating an amount of a S-100 protein and can be measured by a specific radioimmunoassay (e.g., but not limited to Abnova Kit Catalog number KA0037) and result in a 2-fold reduction of a CPB-inflicted serum S-100 protein level elevation. In some embodiments, a reduced CPB-inflicted organ injury can mean a reduction of BBB disruption and can be measured by quantitating an amount of a S-100 protein and can be measured by a specific radioimmunoassay (e.g., but not limited to Abnova Kit Catalog number KA0037) and result in a 1.5-fold-3 fold reduction of a CPB-inflicted serum S-100 protein level elevation. In some embodiments, a reduced CPB-inflicted organ injury can mean a reduction of BBB disruption and can be measured by quantitating an amount of a S-100 protein and can be measured by a specific radioimmunoassay (e.g., but not limited to Abnova Kit Catalog number KA0037) and result in a 2-fold-3 fold reduction of a CPB-inflicted serum S-100 protein level elevation. In some embodiments, a reduced CPB-inflicted organ injury can mean a reduction of BBB disruption and can be measured by quantitating an amount of a S-100 protein and can be measured by a specific radioimmunoassay (e.g., but not limited to Abnova Kit Catalog number KA0037) and result in a 1.5-fold-2 fold reduction of a CPB-inflicted serum S-100 protein level elevation.

In some embodiments, a reduced CPB-inflicted organ injury can mean a reduction of elevation in BAL neutrophil elastase and/or TNF-alpha counts and can be measured by at least one ELISA kit (e.g., but not limited to, Elastase can be measured by use of Abcam Kit Catalog number ab119553; TNF alpha can be measured by use of Life Technologies Kit Catalog Number KHC3011) and result in a 2-fold reduction of neutrophil elastase and TNF-alpha in the alveoli. In some embodiments, a reduced CPB-inflicted organ injury can mean a reduction of elevation in BAL neutrophil elastase and/or TNF-alpha counts and can be measured by at least one ELISA kit (e.g., but not limited to, Elastase can be measured by use of Abcam Kit Catalog number ab119553; TNF alpha can be measured by use of Life Technologies Kit Catalog Number KHC3011) and result in a 1.5-fold-3.0 fold reduction of neutrophil elastase and TNF-alpha in the alveoli. In some embodiments, a reduced CPB-inflicted organ injury can mean a reduction of elevation in BAL neutrophil elastase and/or TNF-alpha counts and can be measured by at least one ELISA kit (e.g., but not limited to, Elastase can be measured by use of Abcam Kit Catalog number ab119553; TNF alpha can be measured by use of Life Technologies Kit Catalog Number KHC3011) and result in a 1.5-fold-2.0 fold reduction of neutrophil elastase and TNF-alpha in the alveoli. In some embodiments, a reduced CPB-inflicted organ injury can mean a reduction of elevation in BAL neutrophil elastase and/or TNF-alpha counts and can be measured by at least one ELISA kit (e.g., but not limited to, Elastase can be measured by use of Abcam Kit Catalog number ab119553; TNF alpha can be measured by use of Life Technologies Kit Catalog Number KHC3011) and result in a 2-fold-3.0 fold reduction of neutrophil elastase and TNF-alpha in the alveoli.

In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 50% reduction of a CPB-inflicted decreased lung oxygen delivery capacity. In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 30-70% reduction of a CPB-inflicted decreased lung oxygen delivery capacity. In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 40-70% reduction of a CPB-inflicted decreased lung oxygen delivery capacity. In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 50-70% reduction of a CPB-inflicted decreased lung oxygen delivery capacity. In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 60-70% reduction of a CPB-inflicted decreased lung oxygen delivery capacity. In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 30-60% reduction of a CPB-inflicted decreased lung oxygen delivery capacity. In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 30-50% reduction of a CPB-inflicted decreased lung oxygen delivery capacity. In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 30-40% reduction of a CPB-inflicted decreased lung oxygen delivery capacity. In some embodiments, a reduced CPB-inflicted organ injury can mean a reduction of decreased lung oxygen delivery capacity as measured by AaDO₂ calculation (AaDO2=(713×FiO2)−(pCO2/0.8)−(paO₂)) and result in a 40-60% reduction of a CPB-inflicted decreased lung oxygen delivery capacity.

In some embodiments, a reduced CPB-inflicted organ injury can mean a reduction of respiratory compromise by a reduction in a decrease of FEV-1 and can be measured by a standard pulmonary function test and results in a 30% reduction of a CPB-inflicted decreased FEV-1 in patients treated with AAT-1. In some embodiments, a reduction in the decrease of FEV-1 can measure between 20-40% in patients treated with AAT-1. In some embodiments, a reduction in the decrease of FEV-1 can measure between 30-40% in patients treated with AAT-1. In some embodiments, a reduction in the decrease of FEV-1 can measure between 20-30% in patients treated with AAT-1.

In some embodiments, a reduced CPB-inflicted organ injury can mean a reduction of respiratory compromise by a reduction in plateau pressure, peak inspiratory pressure, physiologic dead space, and an increase in static compliance, and dynamic compliance. As used herein, “plateau pressure” refers to the pressure applied to small airways and alveoli during positive-pressure mechanical ventilation, and it is measured during an inspiratory pause on the mechanical ventilator.

As used herein, “peak inspiratory pressure” or “PIP” refers to the highest level of pressure applied to the lungs during inhalation. In mechanical ventilation the number reflects a positive pressure in centimeters of water pressure (cmH₂O).

As used herein, “physiologic dead space” refers to the volume of air which is inhaled that does not take part in the gas exchange and can be calculated using the following formula:

$$\frac{{V}_{d}}{{V}_{t}} = \frac{{P}_{{aCO}_{2}} - {P}_{{eCO}_{2}}}{{P}_{{aCO}_{2}}}$$

where V_{d} is the dead space volume and Vₜ is the tidal volume.
   P_{aCO}_{₂} is the partial pressure of carbon dioxide in the arterial blood, and
   P_{eCO}_{₂} is the partial pressure of carbon dioxide in the expired (exhaled) air.

As used herein, “static compliance” or “Cₛₜₐₜ^{”} refers to pulmonary compliance during periods without gas flow, such as during an inspiratory pause, and can be calculated using the formula:

$${C}_{stat} = \frac{{V}_{T}}{{P}_{plat} - PEEP}$$

where Pₚₗₐₜ=plateau pressure. Pₚₗₐₜ is measured at the end of inhalation and prior to exhalation using an inspiratory hold maneuver. During this maneuver, airflow is transiently (˜0.5 sec) discontinued, which eliminates the effects of airway resistance. Pₚₗₐₜ is never >PIP and is typically <3-5 cmH₂O lower than PIP when airway resistance is not elevated.

As used herein, “dynamic compliance” or “C_{dyn}” refers to pulmonary compliance during periods of gas flow, such as during active inspiration. Dynamic compliance is less than or equal to static lunch compliance, and can be calculated using the following equation, where C_{dyn}=Dynamic compliance; VT=tidal volume; PIP=Peak inspiratory pressure (the maximum pressure during inspiration); PEEP=Positive End Expiratory Pressure:

$${C}_{dyn} = \frac{{V}_{T}}{PIP - PEEP} .$$

In some embodiments, a reduced CPB-inflicted organ injury after (e.g., but not limited to, 5 minutes, 10 minutes, 15 minutes, 30 minutes, etc.) a standard cardiac operation of a patient can refer to a reduction in a postoperative rise in plateau pressure of 25% to 10% when compared to a preoperative plateau pressure (e.g., but not limited to, measured 5 minutes, 10 minutes, 15 minutes, 30 minutes prior to operation) of the patient. In some embodiments, a reduced CPB-inflicted organ injury after a standard cardiac operation of a patient can refer to a reduction in a postoperative rise in plateau pressure of 20% to 10% when compared to a preoperative plateau pressure of the patient. In some embodiments, a reduced CPB-inflicted organ injury after a standard cardiac operation of a patient can refer to a reduction in a postoperative rise in plateau pressure of 15% to 10% when compared to a preoperative plateau pressure of the patient. In some embodiments, a reduced CPB-inflicted organ injury after a standard cardiac operation of a patient can refer to a reduction in a postoperative rise in plateau pressure of 25% to 15% when compared to a preoperative plateau pressure of the patient. In some embodiments, a reduced CPB-inflicted organ injury after a standard cardiac operation of a patient can refer to a reduction in a postoperative rise in plateau pressure of 25% to 20% when compared to a preoperative plateau pressure of the patient. In some embodiments, a reduced CPB-inflicted organ injury after a standard cardiac operation of a patient can refer to a reduction in a postoperative rise in plateau pressure of 20% to 15% when compared to a preoperative plateau pressure of the patient.

In some embodiments, a reduced CPB-inflicted organ injury after (e.g., but not limited to, 5 minutes, 10 minutes, 15 minutes, 30 minutes, etc.) a standard cardiac operation of a patient can refer to a reduction in a postoperative rise in peak inspiratory pressure of 25% to 10% when compared to a pre operative peak inspiratory pressure (e.g., but not limited to, measured 5 minutes, 10 minutes, 15 minutes, 30 minutes prior to operation) of the patient. In some embodiments, a reduced CPB-inflicted organ injury after a standard cardiac operation of a patient can refer to a reduction in a postoperative rise in peak inspiratory pressure of 20% to 10% when compared to a pre operative peak inspiratory pressure of the patient. In some embodiments, a reduced CPB-inflicted organ injury after a standard cardiac operation of a patient can refer to a reduction in a postoperative rise in peak inspiratory pressure of 15% to 10% when compared to a pre operative peak inspiratory pressure of the patient. In some embodiments, a reduced CPB-inflicted organ injury after a standard cardiac operation of a patient can refer to a reduction in a postoperative rise in peak inspiratory pressure of 25% to 15% when compared to a pre operative peak inspiratory pressure of the patient. In some embodiments, a reduced CPB-inflicted organ injury after a standard cardiac operation of a patient can refer to a reduction in a postoperative rise in peak inspiratory pressure of 25% to 20% when compared to a pre operative peak inspiratory pressure of the patient. In some embodiments, a reduced CPB-inflicted organ injury after a standard cardiac operation of a patient can refer to a reduction in a postoperative rise in peak inspiratory pressure of 20% to 15% when compared to a pre operative peak inspiratory pressure of the patient. In some embodiments, the peak inspiratory pressure can be assessed repeatedly (e.g., but not limited to, 2, 3, 4, 5, 6, 7, 8, etc. times post operation) during the first 48 post operative hours (e.g., but not limited to, 1 hour post operation, 2 hours post operation, 3 hours post operation, etc.)

In some embodiments, a reduced CPB-inflicted organ injury after (e.g., but not limited to, 5 minutes, 10 minutes, 15 minutes, 30 minutes, etc.) a standard cardiac operation of a patient can refer to a reduction in a postoperative rise in physiologic dead space (VD/VT) of 15% to 5% when compared to a pre-operative physiologic dead space (e.g., but not limited to, measured 5 minutes, 10 minutes, 15 minutes, 30 minutes prior to operation) of the patient. In some embodiments, a reduced CPB-inflicted organ injury after a standard cardiac operation of a patient can refer to a reduction in a postoperative rise in physiologic dead space (VD/VT) of 10% to 5% when compared to a pre-operative physiologic dead space of the patient. In some embodiments, a reduced CPB-inflicted organ injury after a standard cardiac operation of a patient can refer to a reduction in a postoperative rise in physiologic dead space (VD/VT) of 15% to 10% when compared to a pre-operative physiologic dead space of the patient. In some embodiments, the physiologic dead space can be assessed repeatedly (e.g., but not limited to, 2, 3, 4, 5, 6, 7, 8, etc. times post operation) during the first 48 post operative hours (e.g., but not limited to, 1 hour post operation, 2 hours post operation, 3 hours post operation, etc.).

In some embodiments, a reduced CPB-inflicted organ injury after (e.g., but not limited to, 5 minutes, 10 minutes, 15 minutes, 30 minutes, etc.) a standard cardiac operation of a patient can refer to a reduction in a postoperative decline in static compliance of 15% to 5% when compared to a preoperative static compliance (e.g., but not limited to, measured 5 minutes, 10 minutes, 15 minutes, 30 minutes prior to operation) of the patient. In some embodiments, a reduced CPB-inflicted organ injury after a standard cardiac operation of a patient can refer to a reduction in a postoperative decline in static compliance of 10% to 5% when compared to a preoperative static compliance of the patient. In some embodiments, a reduced CPB-inflicted organ injury after a standard cardiac operation of a patient can refer to a reduction in a postoperative decline in static compliance of 15% to 10% when compared to a preoperative static compliance of the patient. In some embodiments, the static compliance can be assessed repeatedly (e.g., but not limited to, 2, 3, 4, 5, 6, 7, 8, etc. times post operation) during the first 48 post operative hours (e.g., but not limited to, 1 hour post operation, 2 hours post operation, 3 hours post operation, etc.).

In some embodiments, a reduced CPB-inflicted organ injury after (e.g., but not limited to, 5 minutes, 10 minutes, 15 minutes, 30 minutes, etc.) a standard cardiac operation of a patient can refer to a reduction in a postoperative decline in dynamic compliance of 10% to 5% (e.g., but not limited to, 5%, 6%, 7%, 8%, etc.) when compared to a preoperative dynamic compliance (e.g., but not limited to, measured 5 minutes, 10 minutes, 15 minutes, 30 minutes prior to operation) of the patient. In some embodiments, the dynamic compliance can be assessed repeatedly (e.g., but not limited to, 2, 3, 4, 5, 6, 7, 8, etc. times post operation) during the first 48 post operative hours (e.g., but not limited to, 1 hour post operation, 2 hours post operation, 3 hours post operation, etc.).

In some embodiments, the percentages noted above regarding the reduced CPB-inflicted organ injury can double in complicated cardiac surgery (e.g., but not limited to, double or triple valve operations, combined valve and coronary artery operations and operations on the aorta) involving long periods of cardiopulmonary bypass machine usage (e.g., but not limited to, 90 minutes or more of usage, e.g., but not limited to, 100 minutes, 120 minutes, etc.).

In some embodiments, a reduced CPB-inflicted organ injury can mean a reduction of respiratory compromise and can be measured by post-operative chest x-ray and result in a 50% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 30-70% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 40-70% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 50-70% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 60-70% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 30-60% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 30-50% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 30-40% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 40-60% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 40-50% reduction of a CPB-inflicted lung atelectasis and/or pleural fluid accumulation. In some embodiments, a reduced CPB-inflicted organ injury can mean a reduction of respiratory compromise and can be measured by post-operative chest x-ray and result in a 50-60% reduction of a CPB-inflicted lung atelectasis and/or pleural fluid accumulation.

In some embodiments, a reduced CPB-inflicted organ injury can mean a reduction of cardiovascular compromise and can be measured by (monitoring cardiac enzyme levels, e.g., CPK and/or Troponin plasma levels) and result in a 30% reduction of a CPB-inflicted serum cardiac enzyme elevation. In some embodiments, a reduced CPB-inflicted organ injury can mean a reduction of cardiovascular compromise and can be measured by (monitoring cardiac enzyme levels, e.g., CPK and/or Troponin plasma levels) and result in a 20-40% reduction of a CPB-inflicted serum cardiac enzyme elevation. In some embodiments, a reduced CPB-inflicted organ injury can mean a reduction of cardiovascular compromise and can be measured by (monitoring cardiac enzyme levels, e.g., CPK and/or Troponin plasma levels) and result in a 30-40% reduction of a CPB-inflicted serum cardiac enzyme elevation. In some embodiments, a reduced CPB-inflicted organ injury can mean a reduction of cardiovascular compromise and can be measured by (monitoring cardiac enzyme levels, e.g., CPK and/or Troponin plasma levels) and result in a 20-30% reduction of a CPB-inflicted serum cardiac enzyme elevation.

In some embodiments, a reduced CPB-inflicted organ injury can mean a reduction of cardiovascular compromise by need and/or magnitude of required inotrope treatment (i.e., an agent which increases or decreases the force or energy of muscular contractions; e.g., but not limited to, cAMP dependent agents (e.g., adrenergic agonists, dopaminergic agonists, phosphodiesterase III isoenzyme inhibitors), cAMP independent inotropic agents (e.g., Na+−K+−ATPase inhibitors, potassium channels inhibitors, agonists of beta-adrenergic receptors, calcium, phenylephrine), additional agents (e.g., calcium sensitizers, vasopressin, natriuretic brain peptide, or any combination thereof) and can be measured by monitoring inotrope usage and result in a 40% reduction of a CPB-inflicted need for postoperative inotrope usage. In some embodiments, an inotrope can be a positive inotrope or a negative inotrope. In some embodiments, an inotrope can be a catecholamine, where the catecholamine can be epinephrine, norepinephrine isoproterenol, noradrenaline, dopamine, dopexamine, dobutamine, levosimendan, PDE inhibitor(s) or any combination thereof. In some embodiments, a reduced CPB-inflicted organ injury can mean a reduction of cardiovascular compromise by need and/or magnitude of required inotrope treatment and can be measured by monitoring inotrope usage and result in a 30-50% reduction of a CPB-inflicted need for postoperative inotrope usage. In some embodiments, a reduced CPB-inflicted organ injury can mean a reduction of cardiovascular compromise by need and/or magnitude of required inotrope treatment and can be measured by monitoring inotrope usage and result in a 40-50% reduction of a CPB-inflicted need for postoperative inotrope usage. In some embodiments, a reduced CPB-inflicted organ injury can mean a reduction of cardiovascular compromise by need and/or magnitude of required inotrope treatment and can be measured by monitoring inotrope usage and result in a 30-40% reduction of a CPB-inflicted need for postoperative inotrope usage.

In some embodiments, a reduced CPB-inflicted organ injury can mean a reduction of low cardiac output syndrome and can be measured by observation for low cardiac output syndrome signs (e.g., but not limited to, a blood pressure reading below 90 in the presence of left atrial pressure above 15) and result in a 40% reduction in the incidence of a CPB-inflicted low cardiac output syndrome. In some embodiments, a reduced CPB-inflicted organ injury can mean a reduction of low cardiac output syndrome and can be measured by observation for low cardiac output syndrome signs (e.g., but not limited to, a blood pressure reading below 90 in the presence of left atrial pressure above 15) and result in a 30-50% reduction in the incidence of a CPB-inflicted low cardiac output syndrome. In some embodiments, a reduced CPB-inflicted organ injury can mean a reduction of low cardiac output syndrome and can be measured by observation for low cardiac output syndrome signs (e.g., but not limited to, a blood pressure reading below 90 in the presence of left atrial pressure above 15) and result in a 40-50% reduction in the incidence of a CPB-inflicted low cardiac output syndrome. In some embodiments, a reduced CPB-inflicted organ injury can mean a reduction of low cardiac output syndrome and can be measured by observation for low cardiac output syndrome signs (e.g., but not limited to, a blood pressure reading below 90 in the presence of left atrial pressure above 15) and result in a 30-40% reduction in the incidence of a CPB-inflicted low cardiac output syndrome.

In some embodiments, a reduced CPB-inflicted organ injury can mean a reduction of cardiovascular compromise and can be measured by decrease of incidence of cardiac arrhythmias as observed by using continuous ECG monitoring and result in a 40% reduction of CPB-inflicted arrhythmia incidence. In some embodiments, a reduced CPB-inflicted organ injury can mean a reduction of cardiovascular compromise and can be measured by decrease of incidence of cardiac arrhythmias as observed by using continuous ECG monitoring and result in a 30-50% reduction of CPB-inflicted arrhythmia incidence. In some embodiments, a reduced CPB-inflicted organ injury can mean a reduction of cardiovascular compromise and can be measured by decrease of incidence of cardiac arrhythmias as observed by using continuous ECG monitoring and result in a 40-50% reduction of CPB-inflicted arrhythmia incidence. In some embodiments, a reduced CPB-inflicted organ injury can mean a reduction of cardiovascular compromise and can be measured by decrease of incidence of cardiac arrhythmias as observed by using continuous ECG monitoring and result in a 30-40% reduction of CPB-inflicted arrhythmia incidence.

In some embodiments, a reduced CPB-inflicted organ injury can mean a reduction of compromised urinary system and can be measured by creatinine levels (e.g., but not limited to, using the Jaffe reaction using alkaline picrate available from CellBioLabs kit catalog number STA-378) and result in a 30% reduction of a CPB-inflicted organ injury. In some embodiments, a reduced CPB-inflicted organ injury can mean a reduction of compromised urinary system and can be measured by creatinine levels (e.g., but not limited to, using the Jaffe reaction using alkaline picrate available from CellBioLabs kit catalog number STA-378) and result in a 20-40% reduction of a CPB-inflicted organ injury. In some embodiments, a reduced CPB-inflicted organ injury can mean a reduction of compromised urinary system and can be measured by creatinine levels (e.g., but not limited to, using the Jaffe reaction using alkaline picrate available from CellBioLabs kit catalog number STA-378) and result in a 30-40% reduction of a CPB-inflicted organ injury. In some embodiments, a reduced CPB-inflicted organ injury can mean a reduction of compromised urinary system and can be measured by creatinine levels (e.g., but not limited to, using the Jaffe reaction using alkaline picrate available from CellBioLabs kit catalog number STA-378) and result in a 20-30% reduction of a CPB-inflicted organ injury.

In some embodiments, a reduced CPB-inflicted organ injury can mean a reduction of kidney injury and can be measured by N-GAL, KIM and/or cystatin C serum levels as measured using at least one ELISA kit (e.g., but not limited to, N-GAL can be measured by use of Enzo Life Sciences Kit catalog number P80188; KIM can be measured by use of Enzo Life Sciences Kit ADI-900-226-0001; cystatin C can be measured by use of Biocat kit catalogue no. 41-CYCHU-E01-AL) and result in a 50% reduction of CPB-inflicted acute kidney injury markers. In some embodiments, a reduced CPB-inflicted organ injury can mean a reduction of kidney injury and can be measured by N-GAL, KIM and/or cystatin C serum levels as measured using at least one ELISA kit (e.g., but not limited to, N-GAL can be measured by use of Enzo Life Sciences Kit catalog number P80188; KIM can be measured by use of Enzo Life Sciences Kit ADI-900-226-0001; cystatin C can be measured by use of Biocat kit catalogue no. 41-CYCHU-E01-AL) and result in a 40-60% reduction of CPB-inflicted acute kidney injury markers. In some embodiments, a reduced CPB-inflicted organ injury can mean a reduction of kidney injury and can be measured by N-GAL, KIM and/or cystatin C serum levels as measured using at least one ELISA kit (e.g., but not limited to, N-GAL can be measured by use of Enzo Life Sciences Kit catalog number P80188; KIM can be measured by use of Enzo Life Sciences Kit ADI-900-226-0001; cystatin C can be measured by use of Biocat kit catalogue no. 41-CYCHU-E01-AL) and result in a 50-60% reduction of CPB-inflicted acute kidney injury markers. In some embodiments, a reduced CPB-inflicted organ injury can mean a reduction of kidney injury and can be measured by N-GAL, KIM and/or cystatin C serum levels as measured using at least one ELISA kit (e.g., but not limited to, N-GAL can be measured by use of Enzo Life Sciences Kit catalog number P80188; KIM can be measured by use of Enzo Life Sciences Kit ADI-900-226-0001; cystatin C can be measured by use of Biocat kit catalogue no. 41-CYCHU-E01-AL) and result in a 40-50% reduction of CPB-inflicted acute kidney injury markers.

The following examples are provided to illustrate certain features and/or embodiments. These examples should not be construed to limit the disclosure to the particular features or embodiments described.

### EXAMPLES

### Example 1: Single Dose Administration of Alpha-1 Anti-Trypsin for Treatment of Organ Injury and Postoperative Bleeding in Patients Undergoing Cardiac Surgery with Cardiopulmonary Bypass

This example describes assay of AAT-1 as an effective inhibitor of injury to a subject undergoing cardiac surgery involving cardiopulmonary bypass. In particular, methods for determining the effect of AAT-1 on postoperative blood loss and organ-function assessment are described.

### Methods

### AAT-1 Dosage

Previous studies and clinical practice indicated that the administration of multiple intravenous AAT-1 doses of 60 mg per kg body weight is safe. Such doing was found result in a low incidence of side-effects, with those reported being benign in nature. Based on pharmacokinetic studies, intraoperative administration of AAT-1 dosage (60 mg/Kg) results in AAT-1 plasma levels which resemble acute phase response; immediately following administration. A 30% reduction in plasma levels is anticipated after termination of CPB with gradual return to normal preoperative AAT-1 levels afterwards.

### Determination of Study Eligibility

Patients eligible in a clinical study to assay use of AAT-1 are male or female, 40-70 years of age. Eligible patents are candidates for isolated coronary artery bypass grafting (CABG) employing cardiopulmonary bypass (CPB), have a calculated logistic Euroscore risk stratification of 5% or less, and will provide signed patient's written informed consent.

For the initial study, exclusion criteria will be based on presence of co-existing conditions including: coagulation abnormalities, severe pulmonary disease defined by blood oxygen saturation of 90% or less or FEV1 of less than 60% of predicted, renal dysfunction defined be serum creatinine levels higher or equal to 1.8 mg %, abnormal liver function tests, uncontrolled diabetes mellitus, severe peripheral vascular disease, a prior cerebrovascular neurological event, abnormal left or right ventricular function, and/or treatment with warfarin or thienopyridine class of anti-platelet agents.

The study participants are randomized to receive either single dose AAT-1 60 mg per kg or placebo.

### Trial Medication Administration

Preparation and dosing of AAT-1 are performed by an unblinded pharmacist. The medication is diluted just prior to administration, and is selected from a commercially available AAT-1 preparation. The patients, research staff, laboratory personnel and data analysts remain blinded to the identity of the treatment from the time of randomization until database lock. Data unblinding is unnecessary. A randomization list is produced by the pharmacist, and was secured and confidential until time of unblinding.

The placebo solution comprises human albumin that resembles the color and consistency of the AAT-1 solution.

The medication is given 3-5 hours prior to surgery (skin incision). Administration rate of the drug does exceed 0.04 ml per kg per minute (for approximately 60-80 minutes). Vital signs including blood pressure, pulse rate and body temperature are correspondingly monitored.

### Surgical Technique

Consistent with study center policy, fentanyl citrate (20-50 mcg/kg), midazolam (2-3 mg) and isoflurane (0.5-2%) are used for induction and maintenance of anesthesia.

Standard median sternotomy is performed followed by harvesting of bypass conduits, uni- or bilateral internal thoracic artery, radial artery or saphenous vein graft. Heparin loading dose is administered prior to initiation of cardiopulmonary bypass (CPB) to achieve kaolin activated coagulation time (ACT). Standard ascending aorta—right atrial cannulation is performed to institute CPB. CPB is initiated after verifying ACT level of 480 seconds or more and periodically monitored. Standard centrifugal pump and a membrane oxygenator are used for extracorporeal circulation (CPB). Consistent with standard technique, active systemic cooling is avoided and patients' core temperature ranges between 32 and 37° C. Distal anastomoses are performed during single aortic cross-clamp and blood cardioplegic arrest. Proximal anastomoses are performed during single aortic cross-clamp. Cold (10° C.) blood cardioplegic solution is delivered in a 4:1 ratio, in antegrade fashion via the aortic root with or without additional retrograde administration via the coronary sinus. After cardioplegic induction (10 ml/Kg) intermittent doses (300-500 ml) are administered; following completion of each distal anastomosis. Heparin is reversed using protamine sulphate in a ratio of 1:1 after weaning from CPB.

### Data Collection

Preoperative Data:

Demographic, morphological and clinical descriptors including age, gender, body mass index (BMI), body surface area (BSA) co-morbidity, Euroscore risk-stratification, medication, etc. is recorded. Preoperative laboratory analysis includes complete blood count, coagulation profile, serum creatinine levels and creatinine clearance, liver function test and arterial blood gases test and serology for HIV, HCV, HBC. Compatible with our routine policy, all patients underwent preoperative echocardiography, coronary angiography, chest x-ray, lung function tests (spirometry) and carotid artery duplex study. Study participants are assigned to undergo preoperative brain MRI; and are subjected to the protocol described below.

Intraoperative:

The type of surgery is categorized. The following data is recorded: heparin dose given prior to bypass initiation; activated clotting time (ACT) counts during the operation (prior, during and after CPB); operative time, cross-clamp time and CPB time; number of trials to wean from CPB, type of inotropes and dosage used during weaning from CPB; blood products (e.g., whole blood (e.g., donated blood), packed cells, fresh frozen plasma, cryoprecipitate, thrombocytes, etc.) utilization during surgery. Allergic reactions or adverse events observed by the surgeon or anesthesiologist are documented. The individual surgeon's impression regarding bleeding tendency is recorded.

### Postoperative Organ Function and Blood Loss Evaluation

The occurrence and magnitude of systemic inflammatory response and organ dysfunction resulting from CPB are recorded and quantified by laboratory markers (e.g., plasma cytokine levels). Related laboratory markers are monitored on a daily basis during the recovery period (in the intensive care unit and at the ward). Specifically, the cytokine levels are monitored during surgery, immediately after surgery (e.g., while the patient is recovering in the intensive care unit) and on a daily basis (e.g., using the blood sampling protocol below). The following organs and corresponding markers are monitored:

Pulmonary Function:

Pulmonary function was evaluated by measured overall mechanical ventilation time, peak inspiratory pressures (PIP), plateau pressures, physiologic dead space and static and dynamic lung compliance. Postoperative dynamic lung compliance was reduced by 20% as compared to preoperative values in untreated patients while no change from preoperative values was observed in the AAT-treated patients. Bronchoalveolar lavage (BAL) was performed 3 hours after operation (while the patient is anesthesized and intubated) and extracted fluid is analyzed for inflammatory markers. Compared to preoperative values, a 6-fold increase of the following markers was observed in untreated patients: Neutrophil elastase, metalloproteinase, TNF α, IL-8, gelatinase, total protein, Neutrophil count. This increase was totally blunted by preoperative AAT-1 administration (e.g., no increase in these markers, i.e. a 0% increase). AaDO₂ calculation [AaDO₂=(713×FiO₂)−(pCO₂/0.8)−(paO₂)] is measured daily. Complete pulmonary function test was performed before and 4 days after the operation. A significantly higher (e.g., 10%, 15%, 20%, 25%, 30%, 35%, 40% higher) FEV1 values were recorded postoperatively in the AAT-1 treated patients. In some embodiments, the FEV1 value was increased by 10-40% in the AAT-1 treated patients. In some embodiments, the FEV1 value was increased by 10-35% in the AAT-1 treated patients. In some embodiments, the FEV1 value was increased by 10-30% in the AAT-1 treated patients. In some embodiments, the FEV1 value was increased by 10-25% in the AAT-1 treated patients. In some embodiments, the FEV1 value was increased by 10-20% in the AAT-1 treated patients. In some embodiments, the FEV1 value was increased by 10-15% in the AAT-1 treated patients. In some embodiments, the FEV1 value was increased by 15-40% in the AAT-1 treated patients. In some embodiments, the FEV1 value was increased by 20-40% in the AAT-1 treated patients. In some embodiments, the FEV1 value was increased by 25-40% in the AAT-1 treated patients. In some embodiments, the FEV1 value was increased by 30-40% in the AAT-1 treated patients. In some embodiments, the FEV1 value was increased by 35-40% in the AAT-1 treated patients. Chest radiographs are evaluated and quantified by a radiologist (e.g., an independent radiologist) for the occurrence of atelectasis, pulmonary edema, or pleural changes (e.g., pleural effusion). The occurrence of either one of these pathologies was significantly lower (e.g., 30% lower) in the AAT-1 treated patients. In some embodiments, the occurrence of atelectasis, pulmonary edema, or pleural changes was 20-40% lower in AAT-1 treated patients. In some embodiments, the occurrence of atelectasis, pulmonary edema, or pleural changes was 30-40% lower in AAT-1 treated patients. In some embodiments, the occurrence of atelectasis, pulmonary edema, or pleural changes was 20-30% lower in AAT-1 treated patients.

Renal Function:

Renal function is evaluated by daily measurements of urine output, serum creatinine levels, creatinine clearance and urinary albumin levels. Acute kidney injury (AKI) markers were sampled in the ICU and these AKI markers include: KIM1, cystatin, N-Gal (sampled 24 hours postoperatively).

Brain Injury Assessment:

The degree of insult to the brain is measured by plasma S-100 protein levels. Assessment of damage to the blood-brain barrier (BBB) was performed by magnetic resonance imaging (MRI) modality on post-operative days 1 and day 5. (see technique protocol below). Each patient underwent at least two standard neuropsychological tests: (1) preoperatively and (2) on postoperative day 3, 4 and/or 5.

Hepatic Function:

Determined by daily measurements of serum hepatic enzymes levels.

Cardiac Function:

Cardiac function is monitored by assaying cardiac enzyme levels; need and magnitude of required inotrope treatment; occurrence of low cardiac output syndrome (defined as systolic blood pressure of 90 mmHg or less coupled with central venous pressure (CVP) of 15 mmHg or more), and incidence of cardiac arrhythmias. Transthoracic echocardiography examination is performed on postoperative day 5 and assessed by a cardiologist (e.g., an independent cardiologist).

Transthoracic echocardiography examination was performed on postoperative day 5 and assessed by an independent cardiologist.

Blood Loss:

Operative and postoperative blood loss is monitored as well as daily hemoglobin levels. Daily platelet counts and thromboelastograms are performed. The distribution of blood products (e.g., but is not limited to, whole blood (e.g., donated blood), packed cells, fresh frozen plasma, cryoprecipitate, thrombocytes, etc.) and total administered are recorded daily. Postoperative CRP levels are evaluated daily.

Blood Sampling and Laboratory Analysis Methods for Cytokine Levels:

10 mL whole blood venous EDTA samples are collected from radial artery catheter at five specified occasions: before induction of anaesthesia, 30 minutes after aortic cross-clamp positioning, and 3, 6, and 9 hours after aortic cross clamp positioning. The blood samples are subsequently centrifuged at 4° C. for 15 min and the serum stored at −70° C. Samples are analyzed for the following cytokines: Polymorphonuclear Neutrophil Elastase (PMNE), Interleukin-1α (IL-1α), Interleukin-1β (IL-1β), Interleukin-2 (IL-2), Interleukin-4 (IL-4), Interleukin-6 (IL-6), Interleukin-8 (IL-8), Interleukin-10 (IL-10), Interferon-γ (IFN-γ), Tumor Necrosis Factor-α (TNF-α), Vascular Endothelial Growth Factor (VEGF), Monocyte Chemoattractant Protein-1 (MCP-1), and Endothelial Growth Factor (EGF). In some embodiments, the levels of cytokines were determined by means of commercially available enzyme-linked immunosorbent assays (ELISA) kits (e.g., but not limited to: IL-6 can be measured by use of Life Technologies Kit Catalog Number KHC0061; TNF alpha can be measured by use of Life Technologies Kit Catalog Number KHC3011; IL-1 beta can be measured by use of Life Technologies Kit Catalog Number KHC0011; IL-8 can be measured by use of Life Technologies Kit Catalog Number KHC0081; MCP-1 can be measured by use of Life Technologies Kit Catalog Number KHC1011; endotoxin can be measured by use of Hyglos GmbH EndoLISA kit Catalog Number 609033; C3a can be measured by use of Enzo Life Sciences Kit Catalog number ADI 900 058; Kinin can be measured by use of Enzo Life Sciences Catalog number ADI-900-206; kalikrein can be measured by use of Enzo Life Sciences Catalog number ADI-900-218-0001; Adhesion molecules can be measured by use of Biotrend Chemikalien GmBh kit Catalog Number E0216Hu-48; Metalloproteinase can be measured by use of Biosensis kit Catalogue No. BEK-2067-2P; elastase can be measured by use of Abcam kit ab119553; Nucleic factor kb can be measured by use of Active Motif kit Catalogue No. 43296; D Dimer can be measured by use of Abbexa kit Catalogue No. abx51360; N-GAL can be measured by use of Enzo Life Sciences Kit P80188; KIM can be measured by use of Enzo Life Sciences kit ADI-900-226-0001; cystatin C can be measured by use of Biocat kit catalogue no. 41-CYCHU-E01-AL).

Daily blood samples are collected postoperatively for platelet count, renal function, liver function, CRP levels S-100 protein and troponin. Post-operative day 1 urine samples for acute kidney injury markers (N-GAL, KIM, Cistatin C) are collected.

Early post-operative adverse events are documented. These include 30-day mortality, new neurological events, myocardial infarction, renal dysfunction, need for re-exploration for bleeding and deep sternal wound infection.

Blood-Brain Barrier (BBB) Assessment by MRI:

The imaging modality used for BBB assessment is through use of a MRI scanner (Philips 3T or General Electric 1.5T). The examination format includes 24 cm FOV, 35 contiguous interleaved slices, 3.5-4 mm thick and co-localized across series. Trace-weighted DWI images are obtained at b=1000 from a 13-15 direction DTI sequence with an in-plane resolution of 2.5×2.5 mm and TR/TE=10s/58 ms at 3T and/or TR/TE=10s/72 ms at 1.5T. T2-FLAIR images are obtained with an in-plane resolution of 0.94×0.94 mm, TR/TE=9000/120 ms and TI=2600 ms at 3T or TR/TE=9000/140 ms and TI=2200 ms at 1.5T.

Method of Measuring Post-Operative Bleeding in a Patient

Postoperative bleeding is quantified by measuring the blood drained (e.g., hourly) through the chest drains. Additionally, the amount of blood and blood products (e.g., but is not limited to, whole blood (e.g., donated blood), packed cells, fresh frozen plasma, cryoprecipitate, thrombocytes, etc.) that the patient receives are monitored.

### Cognitive Testing:

The examples below, i.e., Stroop Color Naming, Proactive Interference, Spatial Task Switching, verbal fluency, and Montreal Cognitive Assessment are all non-limiting examples of methods used to test cognitive function.

Stroop Color Naming will be used to measure inhibition of prepotent response. The Stroop Color Naming method is as follows: a written color name differs from the color ink it is printed in (e.g., the written word says “orange” but the actual color that the word is printed in is “green”), and the participant must say the written word. In the second trial, the participant must name the ink color instead.

Proactive Interference will test a patient's ability (e.g., difficulty) to learn new information because of already existing information. Proactive interference build up occurs with memories being learned in similar contexts. It is also associated with poorer list discrimination, which occurs when participants are asked to judge whether an item has appeared on a previously learned list. If the items or pairs to be learned are conceptually related to one another, then proactive interference has a greater effect. An example of a method of testing is to provide a patient with a list of items to study, and then have the patient recall the items on the list. To further test the patient's ability, add items on the list to recall and/or provide multiple lists to the patient.

Spatial Task Switching examination(s) measure(s) a patient's reactive cognitive flexibility by comparing mixed-task blocks with single task blocks, predictable task-switching and task-cuing paradigms, intermittent instructions, and voluntary task selection. Specifically, the spatial task switching test was performed using the FePsy 2.0 computerized neuropsychological test battery (The Psychology Company P.O. Box 71705 DE 1008 Amsterdam, The Netherlands).

Verbal fluency measures endogenous cognitive flexibility and was performed using the FePsy 2.0 computerized neuropsychological test battery (The Psychology Company P.O. Box 71705 DE 1008 Amsterdam, The Netherlands).

Montreal Cognitive Assessment (MoCA) was performed using the FePsy 2.0 computerized neuropsychological test battery (The Psychology Company P.O. Box 71705 DE 1008 Amsterdam, The Netherlands).

Any other standardized neuropsychological test relevant to the area of BBB disruption may be used to obtain measurements of a patient's cognitive function.

### Results

10 patients are recruited to the study. Five patients receive AAT-1 prior to surgery, and five patients receive placebo. Both groups are comparable with regard to preoperative and operative descriptors (parameters: age, sex, past medical history, left ventricular function, number of grafts performed during surgery and cardiopulmonary bypass time). There are no major operative events and postoperative complications are not observed in the cohort patients. Physical measurements described below are intended as approximations of expected results.

### Brain Injury Assessment

S-100 protein plasma levels were 30% higher in the non-treated patients. Postoperative BBB disruption was seen in 50% of non-treated patients and in only 20% of treated patients. Postoperative decrease in neuropsychological capabilities was recorded in 30% of non-treated patients. None of the treated patients exhibited corresponding decrease.

### Inflammatory Parameters

Intra- and postoperative blood levels of IL-6, TNF-alpha, IL-1 beta, IL-8, MCP-1, LDH, and D dimmer increase in both groups. Elevation of these cytokines is significantly higher in the placebo group.

In patients who did not receive preoperative AAT-1, a significant 3-fold postoperative increase from baseline levels of the following markers (e.g., cytokines) is observed: TNFα, IL-1β, IL-8, MCP-1, D-Dimer IL-6, endotoxin, C3a, kinin, kalikrein, soluble adhesion molecules (e.g., sICAM-1, sVCAM-1, sE-selectin and sP-selectin), metalloproteinase, elastase, nucleic factor Kb. This 3-fold postoperative increase of these markers was reduced and/or obviated by preoperative AAT-1 administration.

Anti-inflammatory cytokine levels (IL-1 receptor antagonist and IL-10) increased by 40% in AAT-1 treated patients. Corresponding levels of IL-1 receptor antagonist and IL-10 are unchanged in patients who are not administered AAT-1.

### Nervous System

MRI at postoperative day (POD) 1 and POD 5 show significant BBB disruption (MR imaging-detected) in 60% of patients in the placebo compared to only 20% in the AAT-1 group. Acute major neurological deficit events are not detected in any patient.

S-100 protein on POD 1 increases by 3-fold in the placebo group compared to 1.5-fold in the AAT-1 group.

### Respiratory System:

Postoperative BAL show significant increase in neutrophil elastase and TNF-alpha counts. Increase of these cytokines is twice more prominent in the placebo group.

Postoperative IL-8 levels decrease more in the placebo group. AaDO₂ decreases in all patients after surgery, more in the placebo group. The AaDO₂ returns to preoperative values on POD 3 in the AAT-1 group compared to POD 5 in the placebo group.

Lung function tests show substantial decrease in FEV-1 and TLC on POD 4 in the placebo group compared to almost no decrease in the AAT-1 group.

Postoperative chest x-rays show atelectasis in 3 of the 5 placebo group and non in the AAT-1 group.

### Cardiovascular System

No signs of low cardiac output syndrome are recorded in any of the patients. Postoperative echocardiography show normal cardiac function in all patients.

Results of monitoring of cardiac enzymes levels showed 30% higher postoperative CPK and Troponin plasma levels in the non-treated patients. Need and magnitude of required inotrope treatment was higher-in the non-treated patients. For example, in AAT-1 treated patients, no inotrope treatment was required, while 2 micrograms per kg per minute of adrenalin was required and administered to patients that had not been treated with AAT-1. Additionally, occurrence of low cardiac output syndrome (defined as systolic blood pressure of 90 mmHg or less coupled with central venous pressure (CVP) of 15 mmHg or more) and incidence of cardiac arrhythmias were both lower in the treated patients (e.g., 10% incidence in patients treated with AAT-1, and 30% incidence in patients not treated with AAT-1). Echocardiographic demonstration of paradoxical septal motion was seen only in the non-treated patients.

In some embodiments, the occurrence of low cardiac output syndrome and incidence of cardiac arrhythmias were 20% reduced in patients treated with AAT-1. In some embodiments, the occurrence of low cardiac output syndrome and incidence of cardiac arrhythmias were 10-30% reduced in patients treated with AAT-1. In some embodiments, the occurrence of low cardiac output syndrome and incidence of cardiac arrhythmias were 20-30% reduced in patients treated with AAT-1. In some embodiments, the occurrence of low cardiac output syndrome and incidence of cardiac arrhythmias were 10-20% reduced in patients treated with AAT-1.

### Urinary System

Average preoperative creatinine is 1.0 mg/dL in both groups of patients. Postoperatively, the average creatinine rises to 1.3 mg/dL in the placebo group and remains 1 mg/dL in the AAT-1 group. Acute kidney injury markers, N-GAL KIM and Cystatin C, increase after surgery in all patients. The increase is substantially higher in the placebo group.

Fluid retention after operation as measured by daily body weight is more prominent in the placebo group (postoperative maximal increase in body weight was twice as much in the placebo group).

### Liver Function

Blood liver enzymes levels increase postoperatively only in the placebo group.

Specifically, daily measurements of serum GOT and GPT levels showed a postoperative 30% elevation of each enzyme (e.g., GOT and/or GPT) only in the patients who were not administered AAT-1.

### Renal Function

All AKI markers were 30% lower in AAT-1 treated patients.

### Post Operative Bleeding and Thrombocytes Function

Operative ACT levels are similar in both groups.

Post-operative bleeding is substantially lower in the AAT-1 group both 6 hours and 24 hours post operatively. Blood D dimmer levels as a marker of fibrinolysis increases more in the placebo group.

Postoperative thromboelastography shows signs of thrombocytes dysfunction in all patients (prolonged K and decreased MA); and more in the placebo group.

The AAT-1 group receives post-operatively about half the amount of blood products (e.g., but is not limited to, whole blood (e.g., donated blood), packed cells, fresh frozen plasma, cryoprecipitate, thrombocytes, etc.) as compared to the placebo group.

Operative bleeding is also assessed by the individual surgeons' impression blinded to the medication and scaled from 1 to 10. The results show that patients in the AAT-1 group tended to bleed less.

### Conclusion

The results of this randomized placebo controlled pilot study indicate that AAT-1 (administered and dosed as described) substantially attenuate CPB-inflicted organ injury. Post-operative bleeding and corresponding need for post-operative blood product (e.g., but is not limited to, whole blood (e.g., donated blood), packed cells, fresh frozen plasma, cryoprecipitate, thrombocytes, etc.) administration are reduced. Administration of AAT-1 also appears to reduce post-CPB inflammation. Hospital length of stay is reduced reflecting improved overall patients' outcome.

### Example 2: Multiple Dose Administration of Alpha-1 Anti-Trypsin (AAT-1) for Treatment of Organ Injury and Post-Operative Bleeding in Patients Undergoing Cardiac Surgery with Cardiopulmonary Bypass

This example describes treatment of post-operative bleeding and organ damage resultant from cardiac surgery by administrations of multiple doses of AAT-1.

Except as specified herein, all methods are as described in Example 1.

As described above, AAT-1 is used to treat or prevent injury resultant from cardiac surgery with cardiopulmonary bypass. Example 1 describes such treatment with a single dose of a composition comprising AAT-1. In the current example, patients are administered two equivalent doses of AAT-1. As described in Example 1, the first dose is administered to the patient as part of the preoperative procedure. Following surgery, the subject is monitored for excessive bleeding and organ injury as described. At post-operative day 1-4, subjects presenting symptoms indicative of excessive bleeding and organ injury are administered a second dose of the composition comprising AAT-1.

### Example 3: Combination Treatment of Organ Injury and Post-Operative Bleeding in Patients Undergoing Cardiac Surgery with Cardiopulmonary Bypass

In this example, damage to a subject resultant from use of cardiopulmonary bypass in cardiac surgery is treated by administering to a subject a combination of AAT-1 and aminocaproic acid.

Methods are as described in the previous examples. In the current example, a subject undergoing cardiopulmonary bypass is administered a composition comprising AAT-1 as part of preoperative treatment. Following surgery, the subject is monitored as described for excessive bleeding and organ damage. A subject presenting symptoms of damage to the respiratory system, urinary system or nervous system is administered a second dose of AAT-1 at 60 mg per kg body weight in a composition containing an effective amount of aminocaproic acid for additional, complimentary treatment.

In view of the many possible embodiments to which the principles of the disclosed invention may be applied, it should be recognized that the illustrated embodiments are only preferred examples of the invention and should not be taken as limiting the scope of the invention.

1-11. (canceled)

12. A method for reducing organ injury inflicted by cardiopulmonary bypass in a subject in need thereof, comprising: administering to said subject a composition comprising an effective amount of alpha-1 antitrypsin (AAT-1), thereby reducing organ injury inflicted by cardiopulmonary bypass in a subject in need thereof.

13. The method of claim 12, wherein said effective amount is 60 mg/kg body weight to 100 mg/kg body weight of said subject.

14. The method of claim 12, wherein said subject: is having a coronary or a cardiac surgery, is destined to be treated with a coronary or a cardiac surgery, undergone a coronary or a cardiac surgery, or any combination thereof.

15. The method of claim 12, wherein said effective amount of AAT-1 is administered intravenously, intranasally, via a cardiopulmonary bypass machine reservoir, or any combination thereof.

16. The method of claim 12, wherein said reducing organ injury inflicted by cardiopulmonary bypass is prophylactically reducing organ injury inflicted by cardiopulmonary bypass.

17. A method for reducing post-operative bleeding in a subject in need thereof, comprising: administering to said subject a composition comprising an effective amount of alpha-1 antitrypsin (AAT-1), thereby reducing post-operative bleeding in a subject in need thereof.

18. The method of claim 17, wherein said effective amount is 60 mg/kg body weight to 100 mg/kg body weight of said subject.

19. The method of claim 17, wherein said subject: is having a coronary or a cardiac surgery, is destined to be treated with a coronary or a cardiac surgery, undergone a coronary or a cardiac surgery, or any combination thereof.

20. The method of claim 17, wherein said effective amount of AAT-1 is administered intravenously, intranasally, via a cardiopulmonary bypass machine reservoir, or any combination thereof.

21. The method of claim 17, wherein said reducing post-operative bleeding is prophylactically reducing post-operative bleeding inflicted by a coronary or a cardiac surgery.


SEQUENCE LISTING (2 sequences)

SEQ ID NO: 1 - DNA, 1000 bp, Homo sapiens
gaattccaggttggaggggcggcaacctcctgccagccttcaggccactctcctgtgcctgccagaagagacagagcttgaggagagcttgaggagagcaggaaaggtggaacattgctgctgctgctcactcagttccacaggtgggaggaacagcagggcttagagtgggggtcattgtgcagatgggaaaacaaaggcccagagaggggaagaaatgcctaggagctaccgagggcaggcgacctcaaccacagcccagtgctggagctgtgagtggatgtagagcagcggaatatccattcagccagctcaggggaaggacaggggccctgaagccaggggatggagctgcagggaagggagctcagagagaaggggaggggagtctgagctcagtttcccgctgcctgaaaggagggtggtacctactcccttcacagggtaactgaatgagagactgcctggaggaaagctcttcaagtgtggcccaccccaccccagtgacaccagcccctgacacgggggagggagggcagcatcaggaggggctttctgggcacacccagtacccgtctctgagctttccttgaactgttgcattttaatcctcacagcagctcaacaaggtacataccgtcaccatccccattttacagatagggaaattgaggctcggagcggttaaacaactcacctgaggcctcacagccagtaagtgggttccctggtctgaatgtgtgtgctggaggatcctgtgggtcactcgcctggtagagccccaaggtggaggcataaatgggactggtgaatgacagaaggggcaaaaatgcactcatccattcactctgcaagtatctacggcacgtacgccagctcccaagcaggtttgcgggttgcacagcggagcgatgcaatctgatttaggcttttaaaggattgcaatcaagtgggacccactagcctcaaccctgtacctcccctcccctccacccccagc

SEQ ID NO: 2 - protein, 417 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKTVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKWNPTQK